SEQUENCE LISTING (6 sequences)

SEQ ID NO: 1 - protein, 498 aa, Homo sapiens [sequence omitted]

SEQ ID NO: 2 - protein, 501 aa, Mus musculus
MAAAAATAAAAAAPSGGGGGGEEERLEEKSEDQDLQGLRDKPLKFKKAKKDKKEDKEGKHEPLQPSAHHSAEPAEAGKAETSESSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLVVKMPFQASPGGKGEGGGATTSAQVMVIKRPGRKRKAEADPQAIPKKRGRKPGSVVAAAAAEAKKKAVKESSIRSVHETVLPIKKRKTRETVSIEVKEVVKPLLVSTLGEKSGKGLKTCKSPGRKSKESSPKGRSSSASSPPKKEHHHHHHHSESTKAPMPLLPSPPPPEPESSEDPISPPEPQDLSSSICKEEKMPRGGSLESDGCPKEPAKTQPMVATTTTVAEKYKHRGEGERKDIVSSSMPRPNREEPVDSRTPVTERVS

SEQ ID NO: 3 - protein, 520 aa, Rattus norvegicus
MAAAAAAAAAAAAAAAAAAAAAAAAPSGGGGGEEERLEEKSEDQDLQGLKEKPLKFKKVKKDKKEDKEGKHEPLQPSAHHSAEPAEAGKAETSESSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTGRPKAAASEGVQVKRVLEKSPGKLLVKMPFQASPGGKGEGGGATTSAQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHAESPKAPMPLLPPPPPPEPQSSEDPISPPEPQDLSSSICKEEKMPRAGSLESDGCPKEPAKTQPMVAAAATTTTTTTTTVAEKYKHRGEGERKDIVSSSMPRPNREEPVDSRTPVTERVS

SEQ ID NO: 4 - protein, 12 aa, Artificial Sequence
  Peptide sequence used as a 1st antigen
GGGEEERLEEKS

SEQ ID NO: 5 - protein, 11 aa, Artificial Sequence
  Peptide sequence used as a 2nd antigen
GGGEEERLEEK

SEQ ID NO: 6 - protein, 13 aa, Artificial Sequence
  Peptide sequence used as a 3rd antigen
GGGEEERLEEKSC

(19) United States
(12) Patent Application Publication
(10) Pub. No.: US 2015/0118235 A1
(43) Pub. Date: Apr. 30, 2015

(54) MeCP2 ISOFORM-SPECIFIC ANTIBODY FOR DETECTION OF ENDOGENOUS EXPRESSION OF MeCP2E1

(71) Applicant: University of Manitoba, Winnipeg (CA)

(72) Inventor: Mojgan Rastegar, Winnipeg (CA)

(73) Assignee: University of Manitoba, Winnipeg (CA)

(21) Appl. No.: 14/062,305

(22) Filed: Oct. 24, 2013

(51) Int. Cl. C07K 16/18; G01N 33/543

(52) U.S. Cl. CPC C07K 16/18; G01N 33/54306; C07K 2317/76; C07K 2317/30; G01N 2333/4703
USPC 424/139.1; 530/389.1; 530/388.1; 530/391.3; 530/327; 435/7.1

(57) ABSTRACT

An antibody that binds a MeCP2E1 isoform of MeCP2 protein, wherein the antibody comprises a region comprising the amino acid sequence of SEQ ID NO:4. A method for detecting and/or monitoring a disease or a disorder caused by an over-expression or an under-expression of a MeCP2E1 isoform of MeCP2 protein, comprising the steps of: (i) obtaining a first sample from a mammalian subject; (ii) contacting the first sample with the anti-MeCP2E1antibody; (iii) removing unbound antibody from the sample; (iv) conducting an immunoassay on the first sample to determine a first value for expression of the MeCP2E1 isoform; (v) comparing the first value to a reference value for expression of the MeCP2E1 isoform in healthy mammalian subjects; wherein a deviation of the first value from the reference value indicates the presence of a disease or a disorder caused by an over-expression or an under-expression of the MeCP2E1 isoform.

### TECHNICAL FIELD

The present disclosure relates generally to compositions and methods for detecting and/or monitoring abnormalities associated with over-expression and under-expression of Methyl CpG Binding Protein 2. More particularly, the present disclosure relates to antibodies or antigen-binding fragments thereof which specifically bind to the MeCP2E1 isoform of Methyl CpG Binding Protein 2, to methods for preparing the antibodies, to compositions containing such antibodies, and to use of the antibodies and/or compositions for detecting the MeCP2E1 isoform of Methyl CpG Binding Protein 2.

### BACKGROUND

Methyl CpG Binding Protein 2 (MeCP2) was discovered in 1992 as a nuclear protein that binds to methylated DNA (Meehan et al., 1992, Characterization of MfeCP2, a vertebrate DNA binding protein with affinity for methylated DNA. Nucleic Acids Res 20:5085-5092). De novo mutations in the X-linked MECP2 gene are associated with more than 90% of reported Rett Syndrome cases (Amir et al., 1999, Rett syndrome is caused by mutations in X-linked MECP2. encoding methyl-CpG-binding protein 2. Nat. Genet. 23:185-188). Rett Syndrome is a severe neurological disorder primarily affecting young females with an incidence of 1 in 10,000 live births. Rett Syndrome patients are mostly asymptomatic up to 6-18 months of age, but start to display impaired locomotor skills, stereotypic hand movements, seizures, abnormal breathing, anxiety and autism. In addition to Rett Syndrome, MECP2 mutations have also been detected in patients with classical autism, X-linked mental retardation, Angelman's syndrome, and severe neonatal encephalopathy.

Alternative splicing of the Mecp2/MECP2 gene leads to the generation of two protein isoforms (i) MeCP2E1 which was previously called MeCP2B or MeCP2α, and (ii) MeCP2E2 which was previously called MeCP2A or MeCP2β (Mnatzakanian et al., 2004. A previously unidentified MECP2 open reading frame defines a new protein isoform relevant to Rett syndrome. Nat. Genet. 36:339-341; Kriaucionis et al., 2004, The major form of MeCP2 has a novel N-terminus generated by alternative splicing. Nucleic Acids Res. 32:1818-1823). MeCP2 protein isoforms differ only in their N-terminal sequences, sharing the same functional Methyl Binding Domain (MBD) and Transcriptional Repression Domain (TRD). This high degree of similarity between the two MeCP2 isoforms suggests that their functional properties might overlap considerably. It appears that that MeCP2E2 is dispensable for Rett Syndrome pathology because selective disruption of Mecp2E2 in mice does not result in the development of Rett Syndrome phenotypes; however, such Rett Syndrome phenotypes have been observed in mice models where both isoforms are disrupted. Accordingly, MECP2E1-specific mutations are sufficient to cause Rett Syndrome while no MECP2E2-specific mutation has been linked to Rett Syndrome thereby shifting the spotlight to MeCP2E1 as the more relevant MeCP2 isoform for Rett Syndrome. In brain, Mecp2/MECP2 isoforms show differential expression with 10× higher expression of the MECP2E1 (Mnatzakanian et al., 2004; Dragich et al., 2007, Differential distribution of the MeCP2 splice variants in the postnatal mouse brain. J. Comp. Neurol. 501:526-542). Whether or not MeCP2E1 protein levels follow the transcript expression in brain is unknown, due to the lack of any available MeCP2 isoform-specific antibodies.

Currently, Rett Syndrome has no effective treatment. However, independent groups have shown that reactivation of the Mecp2 gene after the onset of Rett Syndrome phenotypes in mice, partially rescues physiological and anatomical abnormalities. The problem is that the roles of the two MeCP2 isoforms have not been elucidated because their endogenous expression patterns remain undetermined to date, mainly due to the unavailability of any MeCP2E1 isoform-specific antibody.

### SUMMARY

The exemplary embodiments of the present disclosure relate to antibodies that selectively bind to the MeCP2E1 isoform of the MeCP2 protein, to compositions comprising the anti-MeCP2E1 antibodies, to methods for producing the anti-MeCP2E1 antibodies and compositions comprising the anti-MeCP2E1 antibodies, and to use of the anti-MeCP2E1 antibodies and compositions for detection of and monitoring of the over-expression and/or under-expression of MeCP2E1.

One exemplary embodiment of the present disclosure pertains to methods of preparing anti-MeCP2E1 antibodies or antigen-binding fragments that do not bind to or otherwise engage the MeCP2E2 isoform of the MeCP2 protein. The anti-MeCP2E1 antibodies are generated by a synthetic peptide that consists of a sequence of twelve amino acids selected from the N-terminus of the anti-MeCP2E1 isoform. Alternatively, the anti-MeCP2E1 antibodies may be generated by a synthetic peptide that consists of a sequence of eleven amino acids selected from the N-terminus of the anti-MeCP2E1 isoform.

Another exemplary embodiment of the present disclosure pertains to compositions that include the foregoing antibodies or antigen-binding fragments thereof.

Another exemplary embodiment of the present disclosure pertains to foregoing isolated anti-MeCP2E1 antibodies or antigen-binding fragments thereof packaged in lyophilized form, or packaged in an aqueous medium.

Another exemplary embodiment of the present disclosure pertains to kits for detecting over-expression of MeCP2E1 or under-expression of MeCP2E1 for diagnosis, prognosis or monitoring. The kits include the foregoing isolated anti-MeCP2E1 antibody or antigen-binding fragment thereof labelled with a selected compound, and one or more compounds for detecting the label. Preferably the label is selected from the group consisting of a fluorescent label, an enzyme label, a radioactive label, a nuclear magnetic resonance active label, a luminescent label, and a chromophore label.

Another exemplary embodiment of the present disclosure pertains to methods for detecting an over-expression of MeCP2E1 or an under-expression of MeCP2E1, in a sample from a mammalian subject. The methods include contacting the sample with any of the foregoing antibodies or antigen-binding fragments thereof which specifically bind to a N-terminal domain of MeCP2E1, for a time sufficient to allow the formation of a complex between the antibody or antigen-binding fragment thereof and MeCP2E1, and detecting the MeCP2E1-antibody complex or MeCP2E1-antigen-binding fragment complex. The presence of a complex in the sample is indicative of the presence in the sample of MeCP2E1 or a cell expressing MeCP2E1.

In another aspect, the invention provides other methods for diagnosing a MeCP2E1-mediated disease or disorder in a mammalian subject. The methods include administering to a subject suspected of having or previously diagnosed with MeCP2E1-mediated disease an amount of any of the foregoing antibodies or antigen-binding fragments thereof which specifically bind to an extracellular domain of MeCP2E1 antigen. The method also includes allowing the formation of a complex between the antibody or antigen-binding fragment thereof and MeCP2E1, and detecting the formation of the MeCP2E1-antibody complex or MeCP2E1-antigen-binding fragment antibody complex to the target epitope. The presence of a complex in the subject is indicative of the presence of a MeCP2E1-mediated disease or disorder.

### BRIEF DESCRIPTION OF THE DRAWINGS

The present disclosure will be described in conjunction with reference to the following drawings, in which:

FIG. 1 is a schematic chart of MeCP2 isoforms with known functional domains;

FIG. 2 shows the alignment of MeCP2E1 Isoform 1 amino acid sequences between human, mouse, and rat, and a selected peptide sequence for use as an antigen for generating MeCP2E1 antibodies;

FIG. 3 is a schematic chart of the reported MECP2E1 (Retro-EF1α-E1) and MECP2E2 (Retro-EF1α-E2) retroviral vectors that were used for transfection Pheonix cell lines shown in FIGS. 4 and 5;

FIG. 4 is a Western blot gel of Phoenix cell extracts from control non-transfected (lanes 1, 4), MECP2E1 transfected (lanes 2, 5, 7). MECP2E2 transfected (lanes 3, 6), and MECP2E1 with peptide competition (lane 7). Lanes 1-3 and 7 were probed with anti-MeCP2E1 antibody and lanes 4-6 are probed with anti-MYC antibody, and have been re-probed with anti-ACTIN antibody as a loading control;

FIG. 5 is a Western blot gel of Phoenix cell extracts from non-transfected cells (lane 1), and MECP2E1 transfected cells (lanes 2-5), probed with the anti-MeCP2E1 antibody after pre-incubation with increasing concentrations of peptide (0.1%, 1%, and 5%, as compared to the amount of antibody used to probe the membrane in lanes 3, 4, and 5 respectively);

FIGS. 6(A)-6(D) are micrographs of a single field of view showing immunofluorescence staining of NIH3T3 cells transduced with MECP2E1, wherein FIG. 6(B) shows immunoflourescence with an anti-MeCP2E1 antibody, FIG. 6(C) shows immunoflourescence with an anti-C-MYC antibody, FIG. 6(D) shows DAPI signals, while FIG. 6(A) shows FIGS. 6(B)-6(D) merged together (scale bar represents 10 μm);

FIGS. 7(A)-7(D) are micrographs of a single field of view showing immunofluorescence staining of NIH3T3 cells transduced with MECP2E2 wherein FIG. 7(B) shows lack of immunoflourescence with an anti-MeCP2E1 antibody, FIG. 7(C) shows immunoflourescence with an anti-C-MYC antibody, FIG. 7(D) shows DAPI signals, while FIG. 7(A) shows FIGS. 7(B)-7(D) merged together (scale bar represents 10 μm);

FIGS. 8(A)-8(D) are control micrographs of a single field of view of non-transduced NIH3T3 cells wherein FIG. 8(B) shows lack of immunoflourescence with an anti-MeCP2E1 antibody. FIG. 8(C) shows lack of immunoflourescence with an anti-C-MYC antibody. FIG. 8(D) shows DAPI signals, while FIG. 8(A) shows FIGS. 8(B)-8(D) merged together (scale bar represents 10 μm);

FIGS. 9(A)-9(D) are primary omission control micrographs of a single field of view of MECP2E1-transfected cells wherein FIG. 9(B) shows lack of immunoflourescence with Rhodamine red, FIG. 9(C) shows lack of immunoflourescence with FITC staining, FIG. 9(D) shows DAPI signals, while FIG. 9(A) shows FIGS. 9(B)-9(D) merged together (scale bar represents 10 μm);

FIG. 10(A1) is a micrograph of a tiled image of MeCP2 immunolabelling in an adult mouse hippocampus; FIG. 10(A2) is a micrograph showing immunolabelled MeCP2 in the hippocampus CA1 region; FIG. 10(A3) is a micrograph of the A2 field of view showing immunolabelled MeCP2 with merged signals from DAPI staining; FIG. 10(A4) is a micrograph showing the absence of MeCP2 immunolabelling in the hippocampus CA1 region of Mecp2^{tm1.1Bird} y/− mouse; and FIG. 10(A5) is a micrograph of the A4 field of view showing immunolabelled MeCP2 with merged signals from DAPI staining (scale bar represents 200 μm in A1, and 20 μm in A2-A5);

FIG. 11(B1) is a micrograph of a tiled image of MeCP2E1 immunolabelling in an adult mouse hippocampus; FIG. 11(B2) is a micrograph showing immunolabelled MeCP2E1 in the hippocampus CA1 region; FIG. 11(B3) is a micrograph of the B2 field of view showing immunolabelled MeCP2E1 with merged signals from DAPI staining; FIG. 11(B4) is a micrograph showing the absence of MeCP2E1 immunolabelling in the hippocampus CA region of Mecp2^{tm1.1Bird} y/− mouse; and FIG. 1 (B5) is a micrograph of the B4 field of view showing immunolabelled MeCP2E1 with merged signals from DAPI staining (scale bar represents 200 μm in B1, and 20 μm in B2-B5);

FIG. 12(C1) is a micrograph of a confocal image of MeCP2 immunolabelling in adult mice hippocampus in the CA1 region; FIG. 12(C2) is a micrograph of the C1 field of view additionally showing DAPI nuclear labelling (scale bar represents 2 μm in C1-C2); and FIG. 12(C3) is a chart showing signal intensity profile analysis of two while circles (indicated with the white arrows in C2) indicated that enriched MeCP2 signals are localized at DAPI-rich nuclear regions of cells within the hippocampus CA1;

FIG. 13(D1) is a micrograph of a confocal image of MeCP2 immunolabelling in adult mice hippocampus in the CA1 region; FIG. 13(D2) is a micrograph of the D1 field of view additionally showing DAPI nuclear labelling; and FIG. 13(D3) is a chart showing signal intensity profile analysis of two while circles (indicated with the white arrows in D2) indicated that enriched MeCP2 signals are localized at DAPI-rich nuclear regions of cells within the hippocampus CA1;

FIGS. 14(A) and 14(B) are control micrographs of a single field of view to verify the specificity of MeCP2E1 immunolabelling within the adult murine brain wherein the negative control IgY did not generate any signals in Mecp2^{tm1.1Bird} y/+ mice; while FIGS. 14(C) and 14(D) are control micrographs of a single field of view to verify the specificity of MeCP2E1 immunolabelling within the adult murine brain showing that pre-incubation of the newly generated anti-MeCP2E1 with the antigenic peptide resulted in absence of specific labelling in Mecp2^{tm1.1Bird} y/+ mice (scale bars represent 20 μm);

FIGS. 15(A1) and 15(A2) show the expression of total MeCP2 in the olfactory bulb while FIGS. 15(H1) and 15(H2) show the expression of MeCP2E1 in the olfactory bulb (scale bars represent 80 μm in A1, H1; scale bars represent 20 μmin A2, H2);

FIGS. 16(B1) and 16(B2) show the expression of total MeCP2 in the cerebral cortex while FIGS. 16(I1) and 16(I2) show the expression of MeCP2E1 in the cerebral cortex (scale bars represent 80 μm in B1, I1; scale bars represent 20 μmin B2, I2);

FIGS. 17(C1) and 17(C2) show the expression of total MeCP2 in the while FIGS. 17(J1) and 17(J2) show the expression of MeCP2E1 in the striatum (scale bars represent 80 μm in C1, J1; scale bars represent 20 μmin C2, J2);

FIGS. 18(D1) and 18(D2) show the expression of total MeCP2 in the dentate gyrus while FIGS. 18(K1) and 18(K2) show the expression of MeCP2E1 in the dentate gyrus (scale bars represent 80 μm in D1, K1; scale bars represent 20 μmin D2, K2);

FIGS. 19(E1) and 19(E2) show the expression of total MeCP2 in the thalamus while FIGS. 19(L1) and 19(L2) show the expression of MeCP2E1 in the thalamus (scale bars represent 80 μm in E1, L1; scale bars represent 20 μmin E2, L2);

FIGS. 20(F1) and 20(F2) show the expression of total MeCP2 in the cerebellum while FIGS. 20(M1) and 20(M2) show the expression of MeCP2E1 in the cerebellum (scale bars represent 80 μm in F1, M1; scale bars represent 20 μmin F2, M2);

FIGS. 21(G1) and 21(G2) show the expression of total MeCP2 in the brain stem while FIGS. 21(N1) and 21(N2) show the expression of MeCP2E1 in the brain stem (scale bars represent 80 μm in G1, N1; scale bars represent 20 μmin G2, N2);

FIG. 22 is a chart over two micrographs Western blot gels showing quantification of total MeCP2 in wild type Mecp2^{tm1.1Bird} y/+ mice whole brain (Brain-WT), olfactory bulb, striatum, cerebral cortex, hippocampus, thalamus, brain stem and cerebellum. Mecp2^{tm1.1Bird} y/− mice whole brain (Brain-null) was included as a negative control. Equal loading of protein lysates was verified by probing the same membrane with ACTIN;

FIG. 23 is a chart over two micrographs Western blot gels showing quantification of MeCP2E1 in wild type Mecp2^{tm1.1Bird} y/+ mice whole brain (Brain-WT), olfactory bulb, striatum, cerebral cortex, hippocampus, thalamus, brain stem and cerebellum. Mecp2^{tm1.1Bird} y/− mice whole brain (Brain-null) was included as a negative control. Equal loading of protein lysates was verified by probing the same membrane with ACTIN;

FIGS. 24(A)-24(D) are micrographs showing fluorescence of total MeCP2 immunolabelled embryonic primary cortical neurons labelled with β-III tubulin wherein FIG. 24(B) shows fluorescence of immunolabelled MeCP2; FIG. 24(C) shows fluorescence after labelling with β-III tubulin (β TUB III); FIG. 24(D) shows fluorescence after labelling with DAPI; while FIG. 24(A) shows views 24(B)-24(D) merged together (scale bars represent 5 μm);

FIGS. 25(A)-25(D) are micrographs showing fluorescence of total MeCP2 in immunolabelled embryonic primary astrocytes labelled with GFAP wherein FIG. 25(B) shows fluorescence of immunolabelled MeCP2; FIG. 25(C) shows fluorescence after labelling with GAFP; FIG. 25(D) shows fluorescence after labelling with DAPI; while FIG. 25(A) shows views 25(B)-25(D) merged together (scale bars represent 5 μm);

FIGS. 26(A)-26(E) are micrographs and FIG. 26(F) is a chart showing total MeCP2 signals in embryonic primary cortical neurons display overlapped signals with the constitutive heterochromatin mark H3K9me3 (scale bar represents 2 μm);

FIGS. 27(A)-27(E) are micrographs and FIG. 27(F) is a chart showing total MeCP2 signals in embryonic primary cortical neurons display overlapped signals with the constitutive heterochromatin mark 1H-4K20me3 (scale bar represents 2 μm);

FIGS. 28(A)-28(E) are micrographs and FIG. 28(F) is a chart showing total MeCP2 signals in embryonic primary cortical neurons display signals relative to the facultative heterochromatin mark H3K27me3 (scale bar represents 2 μm);

FIGS. 29(A)-29(E) are micrographs and FIG. 29(F) is a chart showing MeCP2 signals in embryonic primary cortical neurons display signals relative to the facultative heterochromatin mark H3K9me2 (scale bar represents 2 μm);

FIGS. 30(A)-30(D) are micrographs showing fluorescence of MeCP2E1 immunolabelled embryonic primary cortical neurons labelled with NeuN wherein FIG. 30(B) shows fluorescence of immunolabelled MeCP2E1; FIG. 30(C) shows fluorescence after labelling with NEUN; FIG. 30(D) shows fluorescence after labelling with DAPI; while FIG. 30(A) shows views 30(B)-30(D) merged together (scale bars represent 5 μm);

FIGS. 31(A)-31(D) are micrographs showing fluorescence of MeCP2E1 immunolabelled embryonic primary astrocytes labelled with GFAP wherein FIG. 31(B) shows fluorescence of immunolabelled MeCP2E1; FIG. 31(C) shows fluorescence after labelling with GFAP; FIG. 31(D) shows fluorescence after labelling with DAPI; while FIG. 31(A) shows views 31(B)-31(D) merged together (scale bars represent 5 μm); and

FIG. 32 shows micrographs of Western blot analysis of MeCP2E1 levels in primary cortical neurons and astrocytes, while the graph depicts the quantification of MeCP2E1 in neurons and astrocytes.

### DETAILED DESCRIPTION

The exemplary embodiments of the present disclosure pertain to antibodies that selectively bind to the MeCP2E1 isoform of the MeCP2 protein, to compositions comprising the anti-MeCP2E1 antibodies, to methods for producing the anti-MeCP2E1 antibodies and compositions comprising the anti-MeCP2E1 antibodies, and to use of the anti-MeCP2E1 antibodies and compositions for detection of and monitoring of the over-expression and/or under-expression of MeCP2E1.

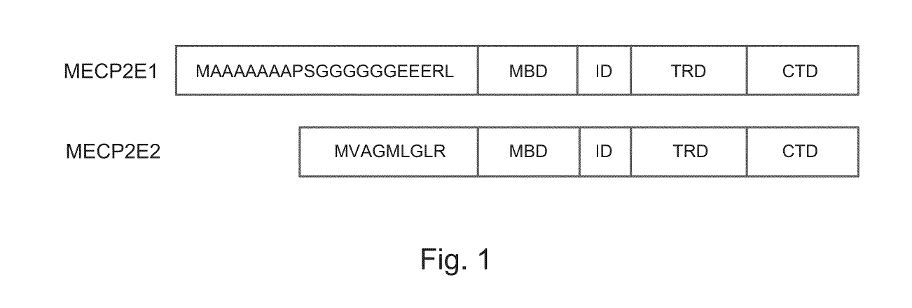

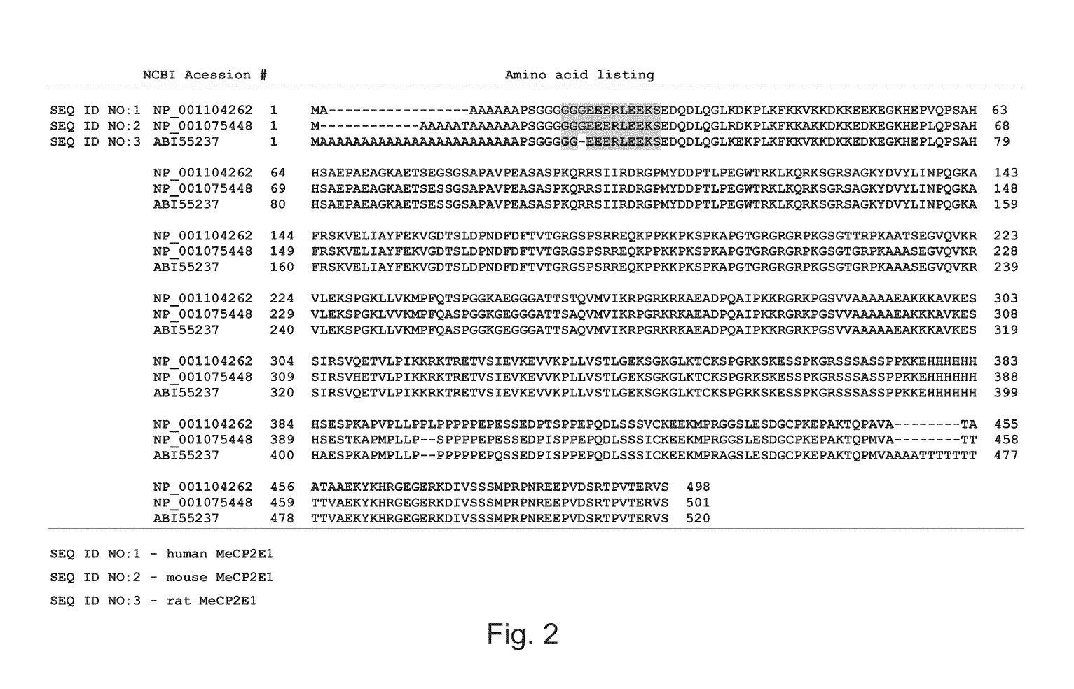

The functional domains of the two MeCP2 isoforms MeCP2E1 and MeCP2E2 are shown in FIG. 1. An amino acid sequence from the N-terminus of the MeCP2E1 isoform (FIG. 2; shaded area) is conserved in human MeCP2E1 protein (FIG. 2; SEQ ID NO:1) and in murine MeCP2E1 protein (FIG. 2; SEQ ID NO:2, SEQ ID NO:3). A peptide sequence consisting of the amino acids “GGGEEERLEEKS” (SEQ ID NO:4) was used as an antigen to produce polyclonal anti-MeCP2E1 antibodies and monoclonal antibodies. A second peptide sequence consisting of the amino acids “GGGEEERLEEK” (SEQ ID NO:5) was also used as an antigen to produce polyclonal anti-MeCP2E1 antibodies and monoclonal antibodies. The anti-MeCP2E1 antibodies produced with the SEQ ID NO:4 and SEQ ID NO:5 antigens selectively bind to and engage the MeCP2E1 protein isoform, but do not bind to the MeCP2E2 protein isoform.

Accordingly, one exemplary embodiment of the present disclosure pertains to methods of preparing anti-MeCP2E1 antibodies or antigen-binding fragments that do not bind to or otherwise engage the MeCP2E2 isoform of the MeCP2 protein. The anti-MeCP2E1 antibodies are generated by a peptide having the amino acid sequence set forth in SEQ ID NO:4. The anti-MeCP2E1 antibodies may also be generated by a peptide having the amino acid sequence set forth in SEQ ID NO:5.

Another exemplary embodiment of the present disclosure pertains to compositions that include the foregoing antibodies or antigen-binding fragments thereof.

Another exemplary embodiment of the present disclosure pertains to foregoing isolated anti-MeCP2E1 antibodies or antigen-binding fragments thereof packaged in lyophilized form, or packaged in an aqueous medium.

Another exemplary embodiment of the present disclosure pertains to kits for detecting over-expression of MeCP2E1 or under-expression of MeCP2E1 for diagnosis, prognosis or monitoring. The kits include the foregoing isolated anti-MeCP2E1 antibody or antigen-binding fragment thereof labelled with a selected compound, and one or more compounds for detecting the label. Preferably the label is selected from the group consisting of a fluorescent label, an enzyme label, a radioactive label, a nuclear magnetic resonance active label, a luminescent label, and a chromophore label.

Another exemplary embodiment of the present disclosure pertains to methods for detecting an over-expression of MeCP2E1 or an under-expression of MeCP2E1, in a sample from a mammalian subject. The methods include contacting the sample with any of the foregoing antibodies or antigen-binding fragments thereof which specifically bind to an extracellular domain of MeCP2E1, for a time sufficient to allow the formation of a complex between the antibody or antigen-binding fragment thereof and MeCP2E1, and detecting the MeCP2E1-antibody complex or MeCP2E1-antigen-binding fragment complex. The presence of a complex in the sample is indicative of the presence in the sample of MeCP2E1 or a cell expressing MeCP2E1.

Another exemplary embodiment pertains to methods for diagnosing a MeCP2E1-mediated disease or disorder in a mammalian subject. The methods include administering to a subject suspected of having or previously diagnosed with MeCP2E1-mediated disease an amount of any of the foregoing antibodies or antigen-binding fragments thereof which specifically bind to an extracellular domain of MeCP2E1 antigen. The method also includes allowing the formation of a complex between the antibody or antigen-binding fragment thereof and MeCP2E1, and detecting the formation of the MeCP2E1-antibody complex or MeCP2E1-antigen-binding fragment antibody complex to the target epitope. The presence of a complex in the subject is indicative of the presence of a MeCP2E1-mediated disease or disorder.

Another exemplary embodiment pertains to use of the anti-MeCP2E1 antibodies or antigen-binding fragments thereof, and/or use of the compositions that include the foregoing antibodies or antigen-binding fragments thereof, and/or use of the kits, and/or use methods for detecting and/or diagnosing Rett's syndrome in a mammalian subject.

Unless defined otherwise, all technical and scientific terms used herein have the same meaning as commonly understood by one of ordinary skill in the art to which the disclosure belongs. Certain terms are discussed in the specification to provide additional guidance to the practitioner in describing the methods, uses and the like of embodiments of the disclosure, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Recital of one or a few synonyms or equivalents does not exclude use of other synonyms or equivalents, unless it is explicitly stated. Use of examples in the specification, including examples of terms, is for illustrative purposes only and does not limit the scope and meaning of the embodiments of the disclosure herein. Although any methods and materials similar or equivalent to those described herein can be used in the practice or testing of the present disclosure, the preferred methods and materials are now described.

To facilitate understanding of the disclosure, the following definitions are provided.

The word “comprise” or variations such as “comprises” or “comprising” will be understood to imply the inclusion of a stated integer or groups of integers but not the exclusion of any other integer or group of integers.

As used herein, the word “complexed” means attached together by one or more linkages. The term “a cell” includes a single cell as well as a plurality or population of cells.

Administering an agent to a cell includes both in vitro administrations and in vivo administrations.

The term “subject” as used herein includes all members of the animal kingdom, and specifically includes humans.

The term “about” or “approximately” means within 20%, preferably within 10%, and more preferably within 5% of a given value or range.

The term “homologous” in all its grammatical forms and spelling variations refers to the relationship between proteins that possess a “common evolutionary origin,” including homologous proteins from different species. Such proteins (and their encoding genes) have sequence homology, as reflected by their high degree of sequence similarity. This homology is greater than about 75%, greater than about 80%, greater than about 85%. In some cases the homology will be greater than about 90% to 95% or 98%.

“Amino acid sequence homology” is understood to include both amino acid sequence identity and similarity. Homologous sequences share identical and/or similar amino acid residues, where similar residues are conservative substitutions for, or “allowed point mutations” of, corresponding amino acid residues in an aligned reference sequence. Thus, a candidate polypeptide sequence that shares 70% amino acid homology with a reference sequence is one in which any 70% of the aligned residues are either identical to, or are conservative substitutions of, the corresponding residues in a reference sequence.

The term “polypeptide” refers to a polymeric compound comprised of covalently linked amino acid residues. Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group. A polypeptide of the disclosure preferably comprises at least about 14 amino acids.

The term “protein” refers to a polypeptide which plays a structural or functional role in a living cell.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

The term “derivative” refers to a product comprising, for example, modifications at the level of the primary structure, such as deletions of one or more residues, substitutions of one or more residues, and/or modifications at the level of one or more residues. The number of residues affected by the modifications may be, for example, from 1, 2 or 3 to 10, 20, or 30 residues. The term derivative also comprises the molecules comprising additional internal or terminal parts, of a peptide nature or otherwise. They may be in particular active parts, markers, amino acids, such as methionine at position −1. The term derivative also comprises the molecules comprising modifications at the level of the tertiary structure (N-terminal end, and the like). The term derivative also comprises sequences homologous to the sequence considered, derived from other cellular sources, and in particular from cells of human origin, or from other organisms, and possessing activity of the same type or of substantially similar type. Such homologous sequences may be obtained by hybridization experiments. The hybridizations may be performed based on nucleic acid libraries, using, as probe, the native sequence or a fragment thereof, under conventional stringency conditions or preferably under high stringency conditions.

The term “antibody” as used herein refers to a glycoprotein comprising at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, C_{H}1, C_{H}2 and C_{H}3. Each light chain is comprised of a light chain variable region (abbreviated herein as LCVR or V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody as used herein, refers to one or more portions of an antibody that retain the ability to specifically bind to an antigen (e.g., MeCP2E1 isoform of the MeCP2 protein). It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546) which consists of a V_{H} domain; and (vi) an isolated complementarity determining region (CDR). Furthermore, although the two domains of the Fv fragment, V and V_{H}, are coded for by separate genes, they can be joined, using recombinant methods, by a synthetic linker that enables them to be made as a single protein chain in which the V₁, and Vᵤ regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983). The fragments are screened for utility in the same manner as are intact antibodies.

An “isolated antibody”, as used herein, is intended to refer to an antibody which is substantially free of other antibodies having different antigenic specificities (e.g., an isolated antibody that specifically binds to MeCP2E1 isoform of the MeCP2 protein is substantially free of antibodies that specifically bind antigens other than the MeCP2E1 isoform). As used herein, “specific binding” refers to antibody binding to a predetermined antigen. Typically, the antibody binds with an affinity that is at least two-fold greater than its affinity for binding to a non-specific antigen (e.g., BSA, casein) other than the predetermined antigen or a closely-related antigen.

The term “complementarity determining regions” as used herein refers to the regions within antibodies where these proteins complement an antigen's shape. The acronym CDR is used herein to mean “complementarity determining region”.

The antibodies of the present disclosure may be polyclonal antibodies and can be produced by a variety of techniques well known in the art. Procedures for raising polyclonal antibodies are well known. For example anti-MeCP2E1 polyclonal antibodies are raised by administering a synthetic peptide (e.g., SEQ ID NO:4) subcutaneously to New Zealand white rabbits which have first been bled to obtain pre-immune serum. The synthetic peptide can be injected at a total volume of 100 μl per site at six different sites, typically with one or more adjustments. The rabbits are then bled two weeks after the first injection and periodically boosted with the same antigen three times every six weeks. A sample of serum is collected 10 days after each boost. Polyclonal antibodies are recovered from the serum, preferably by affinity chromatography using the synthetic peptide to capture the antibody. This and other procedures for raising polyclonal antibodies are disclosed in E. Harlow, et. al., editors, Antibodies: A Laboratory Manual (1988), which is hereby incorporated by reference.

An embodiment of the present disclosure relates to a method of detecting cells or portions thereof in a biological sample (e.g., histological or cytological specimens, biopsies and the like) wherein the MeCP2E1 is overexpressed. This method involves providing an antibody or an antigen-binding binding fragment thereof, probe, or ligand, which specifically binds to an a peptide having a sequence with at least 90% with SEQ ID NO:4 or alternatively with SEQ ID NO:5, preferably at least about 95% identical, more preferably at least about 97% identical, still more preferably at least about 98% identical, and most preferably is at least about 99% identical. The anti-MeCP2E1 antibody is bound to a label that permits the detection of the cells or portions thereof upon binding of the anti-MeCP2E1 antibody to the cells or portions thereof. The biological sample is contacted with the labeled anti-MeCP2E1 antibody under conditions effective to permit binding of the anti-MeCP2E1 antibody to the N-terminal domain of MeCP2E1 of any of the cells or portions thereof in the biological sample. The presence of any cells or portions thereof in the biological sample is detected by detection of the label. In one preferred form, the contact between the anti-MeCP2E1 antibody and the biological sample is carried out in a living mammal and involves administering the anti-MeCP2E1 antibody to the mammal under conditions that permit binding of the anti-MeCP2E1 antibody to MeCP2E1 of any of the cells or portions thereof in the biological sample. Again, such administration can be carried out by any suitable method known to one of ordinary skill in the art.

In addition, the anti-MeCP2E1 antibodies of the present disclosure can be used in immunofluorescence techniques to examine human tissue, cell and bodily fluid specimens. In a typical protocol, slides containing cryostat sections of frozen, unfixed tissue biopsy samples or cytological smears are air dried, formalin or acetone fixed, and incubated with the monoclonal antibody preparation in a humidified chamber at room temperature. The staining pattern and intensities within the sample are then determined by fluorescent light microscopy and optionally photographically recorded.

As yet another alternative, computer enhanced fluorescence image analysis or flow cytometry can be used to examine tissue specimens or exfoliated cells, i.e., single cell preparations from aspiration of tissues or organs using the anti-MeCP2E1 antibodies of this disclosure. The percent MeCP2E1 positive cell population, alone or in conjunction with determination of other attributes of the cells (e.g., DNA ploidy of these cells), may, additionally, provide very useful prognostic information by providing an early indicator of disease progression.

The method of the present disclosure can be used to screen patients for diseases or disorders associated with the over-expression or under-expression of MeCP2E1. Alternatively, it can be used to identify the recurrence of such diseases or disorders, particularly when the disease or disorder is localized in a particular biological material of the patient.

Also within the scope of the disclosure are kits comprising the compositions of the disclosure and instructions for use. Kits containing the antibodies or antigen-binding fragments thereof of the present disclosure can be prepared for in vitro diagnosis, prognosis and/or monitoring of the over-expression or the under-expression of by the immunohistological, immunocytological and immunoserological methods described above. The components of the kits can be packaged either in aqueous medium or in lyophilized form. When the antibodies or antigen-binding fragments thereof are used in the kits in the form of conjugates in which a label moiety is attached, such as an enzyme or a radioactive metal ion, the components of such conjugates can be supplied either in fully conjugated form, in the form of intermediates or as separate moieties to be conjugated by the user or the kit.

A kit may comprise a carrier being compartmentalized to receive in close confinement therein one or more container means or series of container means such as test tubes, vials, flasks, bottles, syringes, or the like. A first of said container means or series of container means may contain one or more anti-MeCP2E1 antibodies or antigen-binding fragments thereof or MeCP2E1. A second container means or series of container means may contain a label or linker-label intermediate capable of binding to the primary anti-MeCP2E1 antibodies (or fragment thereof).

The present disclosure will be further elaborated in the following examples. However, it is to be understood that these examples are for illustrative purposes only, and should not be used to limit the scope of the present disclosure in any manner.

### EXAMPLES

### Example 1

### MeCP2E1 Antibody Generation

The peptide sequence GGGEEERLEEKS (SEQ ID NO:4; shaded section in FIG. 2) from the N-terminus of MeCP2E1 isoform that is conserved in murine and human MeCP2 protein was selected as the antigen for polyclonal antibody production in chicken. The IgY molecules were purified from chicken egg yolks and anti-MeCP2E1-specific immunoglobulins were isolated by peptide affinity purification.

An additional “C” residue was inserted at the N-terminal end of the sequence GGGEEERLEEKSC (SEQ ID NO:6). The additional C (underlined) was used for conjugation with BSA (Bovine Serum Albumin) and KLH (Keyhole limpet hemocyanin) for antibody purification. The generated antibodies were tested against MeCP2E1 peptide by ELISA during production, and were also tested by WB (Western Blot) and immunofluorescence (IF) studies in transfected Phoenix cells and transduced NIH3T3 cells with MECP2E1 retroviral vectors carrying human MECP2E1 cDNA. The overexpressed MeCP2E1 has a MYC tag and we confirmed the detection of similar signals with C-MYC antibody. Importantly, C-MYC also detects MeCP2E2 (the other MeCP2 isoform that has a MYC tag). However, the anti-MeCP2E1 antibody disclosed herein does not cross-react with the MeCP2E2 isoform.

In addition to testing the antibodies during production, the specificity of the purified anti-MeCP2E1 antibodies was tested and validated by WB and IF studies. Additionally, detection of endogenous mouse MeCP2E1 protein by the purified anti-MeCP2E1 antibody was confirmed by WB, IF and immunohistochemistry (IHC) experiments. All required control experiments showed the specificity of this newly generated anti-MeCP2E1 antibody against the endogenous protein with no cross-reactivity with the other isoform (MeCP2E2).

Additionally, mouse monoclonal antibodies were generated against MCCP2E1 with the same strategy and the corresponding clones were selected based on positive ELISA readings. It was confirmed that these monoclonal antibodies detect exogenous MeCP2E1 by WB and IF.

Our data shows that inclusion or exclusion of an extra amino acid at the N-terminal part of the peptide does not affect the specificity of the generated antibody and does not cause any cross-reactivity with the other MeCP2E2 isoform. Therefore, both peptides: GGGEEERLEEKS (SEQ ID NO:4) or GGGEEERLEEK (SEQ ID NO: 5) can be successfully used for generating polyclonal or monoclonal antibodies against MeCP2E1 protein isoform.

### Example 2

### Generation of MECP2E1/E2 Transfected/Transduced Cells

Retro-EF1α-E1 (expressing MECP2E1) and Retro-EF1α-E2 (expressing MECP2E2) vectors were transfected into Phoenix retroviral packaging cells (Kinsella et al., 1996, Episomal vectors rapidly and stably produce high-titer recombinant retrovirus. Hum. Gene. Ther 0.7:1405-1413.) following the method taught by Rastegar et al. (2009, MECP2 isoform-specific vectors with regulated expression for Rett syndrome gene therapy. PLoS One 4: e6810) to generate (i) infectious retroviral MECP2E1 vectors with a C-terminal C-Myc tag particles, and (ii) retroviral MECP2E2 vectors with a C-terminal C-Myc tag particles. Culture supernatants containing viral particles were harvested at 48 hours post-transfections. The transfected phoenix cells were collected and lysed for protein extraction, and the retroviral particles were used to transduce NIH3T3 mouse fibroblasts following the method taught by Rastegar et al. (2009). The transduced cells were fixed with 4% paraformaldehyde for immunofluorescent studies, 48 hours after transduction. NIH2T3 cells, Phoenix cells, and MECP2 vectors were obtained from The Hospital for Sick Children, Toronto, ON, CA.

### Example 3

### Isolation of Primary Neurons and Astrocytes

Post-mitotic cortical neurons were isolated from embryonic day 18 (E18) mouse embryos from a CD1 background following the method taught by Rastegar et al. (2009). Briefly, cerebral cortices were dissected from E18 mouse embryos, dissociated using papain, and then triturated with a fire-polished Pasteur pipette. Subsequently, the cells were resuspended in NEUROBASAL medium with B-27 supplement (NEUROBASAL is a registered trademark of Life Technologies Inc., Carlsbad, Calif., USA) and plated at a density of 1.2×10⁵ cells/ml in poly-lysine coated dishes. After three days, 50% of the media was replaced with fresh medium. Subsequently, media was replenished every 48 hours. Cells were lysed/fixed 7 days after seeding for further experiments.

Primary cortical astrocytes were isolated from E18 mouse embryos from a CD1 background following the method taught by Shao et al. (2009, Functional and immunocytochemical characterization of D-serine transporters in cortical neuron and astrocyte cultures. J. Neurosci. Res. 87:2520-2530). Briefly, cerebral cortices were isolated from E18 mouse embryos and dissociated using papain. The cells were triturated using narrow-ended pipettes and resuspended in Minimal Essential Medium (MEM; Sigma Aldrich, Oakville, ON, CA) with 10% FBS. Subsequently, the cells were seeded at a density of 2×10⁵ cells/ml in poly-lysine coated dishes. Media was replaced every 48 hours. Cells were lysed/fixed 14 days after seeding for further experiments.

### Example 4

### Immunohistochemistry, Immunofluorescence and Fluorescent Imaging

WT Mecp2^{tm1.1Bird} y/+ mice and null Mecp2^{tm1.1Bird} y/− mice in the C57/BL 6 background. were purchased from The Jackson Laboratory (Bar Harbor, Me., USA). Additional C57/BL 6 WT were obtained from central animal care services at the University of Manitoba (Winnipeg, MB, CA). Brain tissues were isolated from euthanized mice (C57/BL 6), cut into small blocks and incubated in freshly de-polymerized paraformaldehyde fixative solution (0.16M sodium phosphate buffer, pH7.4 with PFA) for 20 minutes (min) and rinsed with cryoprotectant solution (25 mM sodium phosphate buffer, pH7.4, 10% sucrose, 0.04% NaN3) for immunohistochemistry (IHC) studies. Subsequently, the tissue blocks were incubated in cryoprotectant at 4° C. for approximately 24 h. Cryosections were processed on to gelatinized slides and stored at −20° C. Prior to IHC experiments, slides were air-dried and permeabilized for 20 min with 0.3% Triton X-100 Tris-buffered saline (TBS-Tr) (50 mM Tris-HCl, pH 7.4, containing 1.5% NaCl) solution. The slides were then pre-blocked with 20% normal donkey serum (NDS, Jackson Immunoresearch Laboratories Inc., West Grove, Pa., USA) in TBS-Tr and incubated with appropriate primary antibodies diluted in 10% NDS in TBS-Tr overnight at 4° C. Primary antibody incubation was followed by three washes with TBS-Tr. Secondary antibodies in diluted TBS-Tr/10% NDS were applied for 1 h at room temperature, followed by one wash with TBS-Tr and two washes using Tris-HCl buffer (50 mM, pH 7.4). The slides were then mounted on Prolong Gold antifade containing 2 μg/ml 4′,6-diamidino-2-phenylindole (DAPI) (EMD Millipore, Billerica, Mass., USA) counter-stain.

For immunofluorescence (IF) studies, cells were fixed with 4% formaldehyde for 10 minutes on ice and processed following the method taught by Rastegar et al. (2009). The coverslips were then slide-mounted with anti-fade medium containing DAPI (0.5 μg/ml). Immunolabelled signals were detected using a ZEISS® AXIO® Observer Z 1 inverted microscope and LSM710 Confocal microscope (Carl Zeiss Canada Ltd, Toronto, ON, CA; ZEISS and AXIO are registered trademarks of Carl Zeiss AG Corp., Oberkochen, Fed. Rep. Ger.). Images were obtained using AXIOVISION® 4.8 (AXIOVISION is a registered trademark of Carl Zeiss AG Corp.), Zen Blue 2011, Zen Black 2009 and Zen Black 2011 softwares (Carl Zeiss Canada Ltd) and assembled using ADOBE® PHOTOSHOP® CS5 and ADOBE® ILLUSTRATOR® CS5 (ADOBE, PHOTOSHOP, and ILLUSTRATOR are registered trademarks of Adobe Systems Inc., San Jose, Calif., USA).

### Example 5

### Western Blotting

Total cell extracts were prepared and Western blotting (WB) was done following the methods taught by Rastegar et al. (2009). 2 μg of total protein extracts from transfected cells or 100 μg of total cell extracts from brain, primary neurons or astrocytes were loaded into each lane and were subjected to WB analysis. All probed membranes were subjected to a second WB with an anti-ACTIN antibody as a loading control. Quantification of detected MeCP2 or MeCP2E1 bands was done with ADOBE® PHOTOSHOP® CS5 software and all bands were normalized to ACTIN signals. Student's t-test was used to analyze the significance of MeCP2 protein levels between samples. For peptide incubation experiments, increasing amounts of peptide antigen (as compared to the antibody concentration) was pre-incubated with the antibody for 3-5 hours at 4° C. before probing the membrane.

### Example 6

### Antibodies

The following antibodies were used in this study: mouse monoclonal anti-MeCP2 (ab50005, WB-1:1000; IF-1:200), rabbit polyclonal anti-H3K9me3 (ab8898, IF-1:200), rabbit polyclonal anti-H4K20me3 (ab9053, IF-1:200), mouse monoclonal anti-H3K27me3 (ab6002, IF-1:200), mouse monoclonal anti-H3K9me2 (ab1220, IF-1:200) (Abcam PLC, San Francisco, Calif., USA); rabbit polyclonal anti-MeCP2 (07-013. WB-1:1000, IF-1:200), mouse monoclonal anti-β-Tubulin III (MAB1637; WB-1:1000, IF-1:200), mouse monoclonal anti-NeuN (MAB377, IF-1:200), chicken polyclonal anti-β-Tubulin III (AB9354, IF-1:200) (EMD Millipore, Billerica, Mass., USA); mouse monoclonal anti-C-MYC (A21280, WB-1:1000, IF-1:200), mouse monoclonal anti-GFAP (A21282, IF-1:200) (Molecular Probes, Eugene, Oreg., USA); mouse monoclonal anti-ACTIN (A2228, WB-1:2500) (Sigma Aldrich, Oakville, ON, CA).

The following secondary antibodies were used: Gt anti-rabbit Alexa Fluor 488 (A11034, IF-1:1000), Gt anti-rabbit Alexa Fluor 594 (A11037, IF-1:1000), Gt anti-chicken Alexa Fluor 594 (A11042, IF-1:1000) (Molecular Probes, Eugene, Oreg., USA); Gt anti-rabbit Rhodamine Red-X (111-295-144, IF-1:400), Gt anti-chicken Rhodamine Red-X (103-295-155, IF-1:400), Peroxidase-Affinipure Gt anti-mouse IgG (115-035-174; WB-1:7500), Perox-AffiniPure Dnk anti-rabbit IgG (711-035-152; WB-1:400) (Jackson Immunoresearch Laboratories Inc., West Grove, Pa., USA).

### Example 7

### Generation of an Anti-MeCP2E1 Antibody and Validation In Vitro

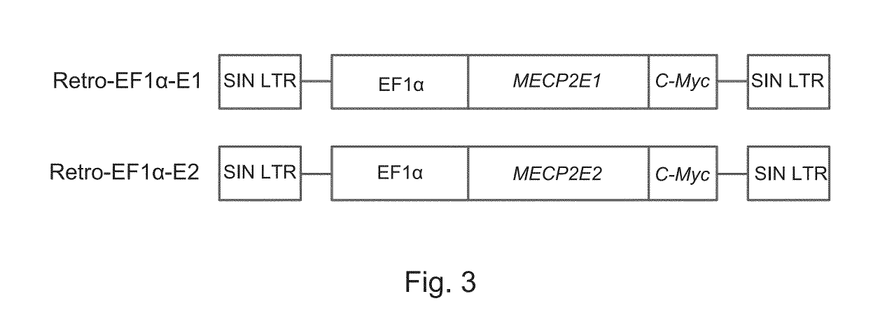

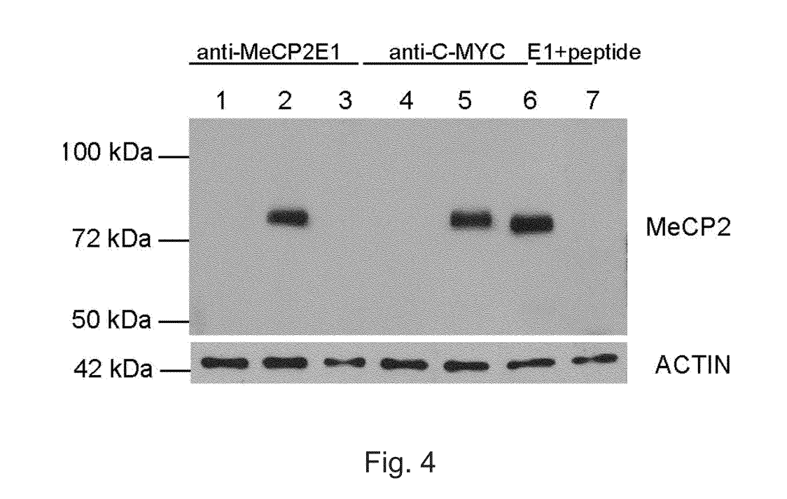

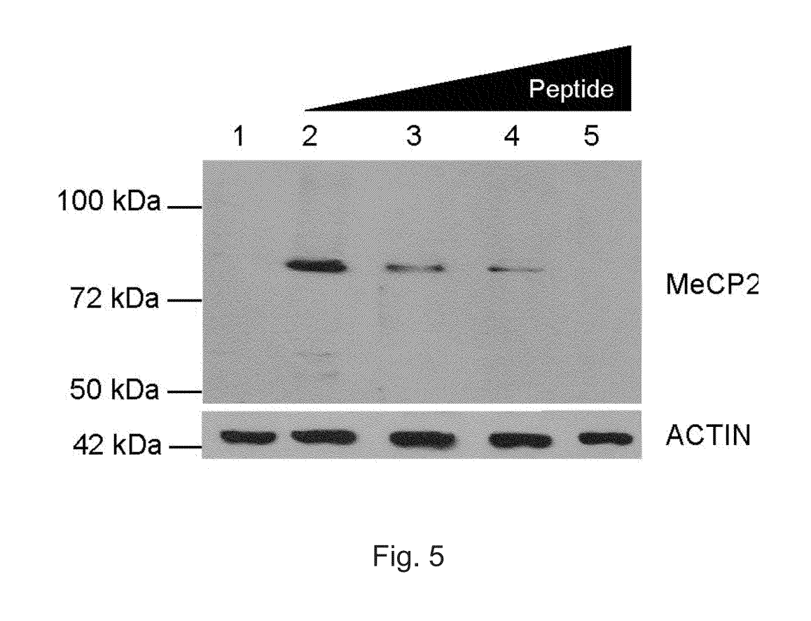

Polyclonal chicken anti-MeCP2E1 antibodies were generated using two synthetic peptides (SEQ ID NO:4; SEQ ID NO:5) spanning part of the N-terminal region of MeCP2E1 (FIGS. 1, 2). Specificity of this anti-MeCP2E1 antibody was validated by WB and IF experiments throughout the course of antibody production and after IgY purification. For WB application, the purified antibody was tested using cell extracts from Phoenix cells transfected with either Retro-EF1α-E1 or Retro-EF1α-E2 (FIG. 3), in parallel to non-transfected control cells. As expected, WB analysis with the anti-MeCP2E1 antibody yielded a specific band at the expected molecular weight (slightly higher than 72 kDa) in MeCP2E1-transfected cells (FIG. 4, lane 2). In contrast, no bands were detected in non-transfected cells (FIG. 4, lane 1), nor in the transfected cells with MECP2E2 (FIG. 4, lane 3). Importantly, pre-incubation of the anti-MeCP2E1 antibody with the antigenic peptide used to generate the antibody (peptide competition) eliminated the detected band in MeCP2E1 transfected cells (FIG. 4, lane 7). The presence of exogenous MeCP2 in the transduced cells with either Retro-EF1α-E1 or Retro-EF1α-E2 was confirmed by immunolabelling with an anti-C-MYC antibody (FIG. 4, lanes 5, 6), with no detectable signal in non-transfected cells (FIG. 4, lane 4). The specificity and sensitivity of the newly developed antibody was further verified by pre-incubation of the anti-MeCP2E1 antibody with increasing concentrations of the antigenic peptide before probing the membranes with MECP2E1 transfect cell lysates (FIG. 5, lanes 2-5). Non-stransfected Phoenix cell lysates were used as a negative control (FIG. 5, lane 1).

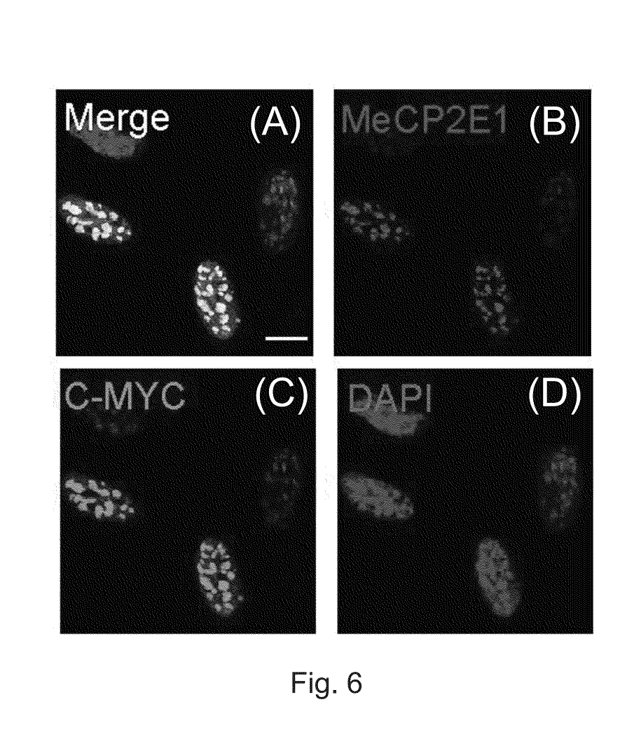

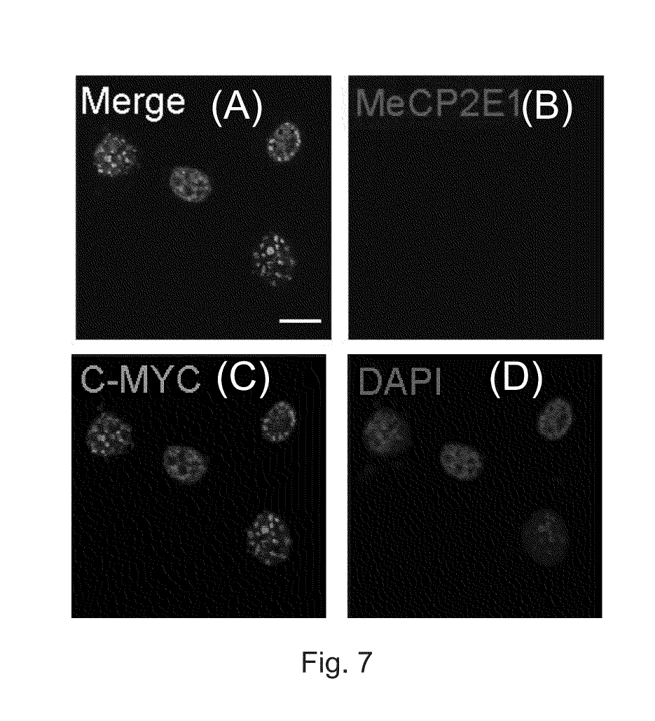

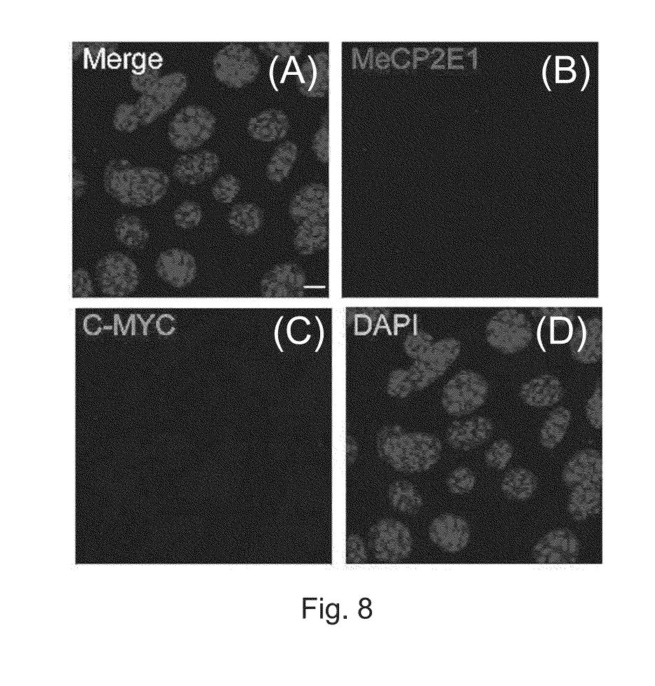

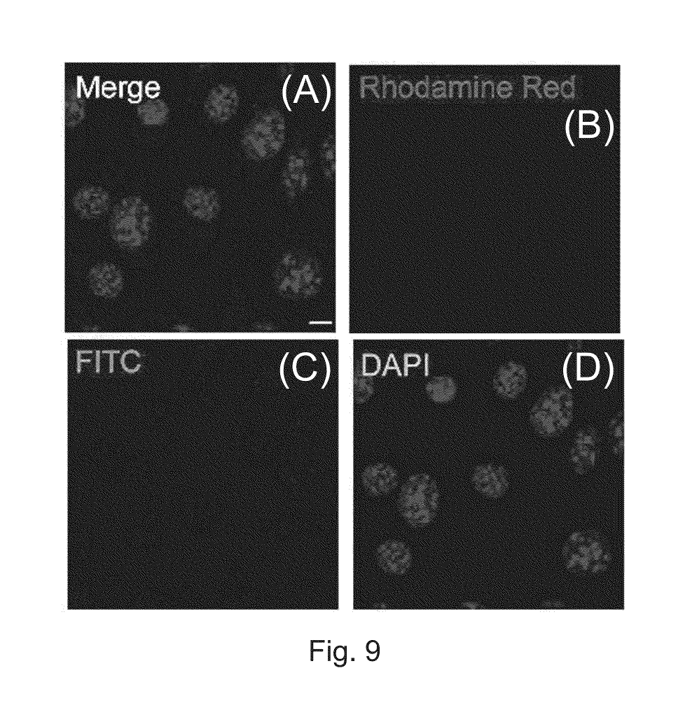

IF staining with the anti-MeCP2E1 antibody revealed the expression of MeCP2 in the DAPI-rich heterochromatic foci within the NIH3T3 cells transduced with MeCP2E1 (FIGS. 6(A)-6(D)), but no signal was detected in MECP2E2-transduced cells (FIGS. 7(A)-7(D)) indicating that that the newly developed antibody does not cross-react with the overexpressed MeCP2E2. In both MeCP2E1-overexpressed cells and MeCP2E2-overexpressed cells, incubation with an anti-C-MYC antibody resulted in detectable signals indicating the protein is properly expressed in both types if cells (FIGS. 6(D) and 7(D)). The absence of endogenous MeCP2E1 expression was confirmed in the non-transduced NIH3T3 cells using the anti-MeCP2E1 antibody (FIG. 8). As expected, no signals were in primary omission experiments using Retro-EF1α-E1 transduced cells with the same secondary antibody (FIG. 9).

These results demonstrate that the anti-MeCP2E1 antibody generated using the synthetic peptide (SEQ ID NO:4) specifically detects MeCP2E1 protein, and shows no cross-reactivity with MeCP2E2.

### Example 8

### The Anti-MeCP2E1 Antibody Generated with the Synthetic Peptide (SEQ ID NO:4) Shows Specificity in Detecting Endogenous MeCP2E1 in Mice

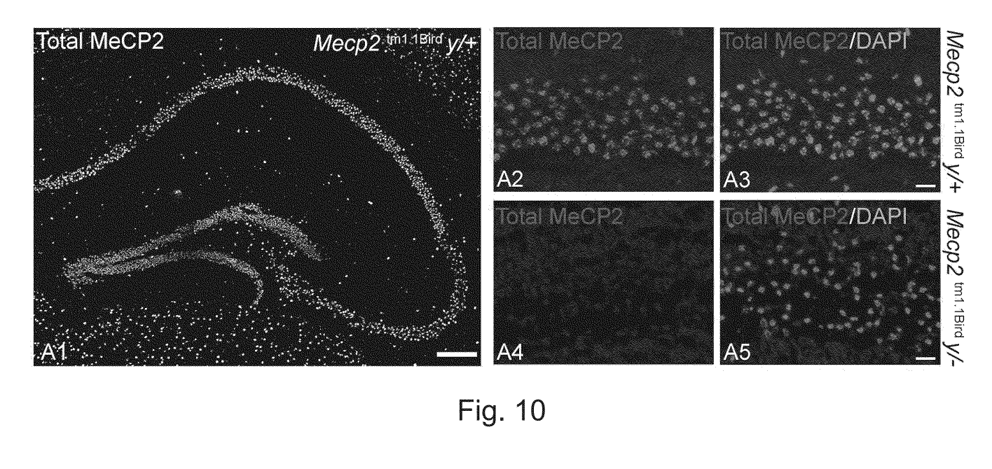

In order to investigate whether the anti-MeCP2E1 antibody generated with the synthetic peptide (SEQ ID NO:4) is capable of detecting the endogenous MeCP2E1, the specificity of this antibody in murine adult brain was tested. Previous studies have reported that MeCP2E1 is highly expressed in murine adult brain (Shahbazian et al., 2002, Insight into Rett syndrome: MeCP2 levels display tissue- and cell-specific differences and correlate with neuronal maturation. Hum. Mol. Genet. 11:115-124). In agreement with these reports, immunohistochemical (IHC) experiments using a commercial anti-MeCP2 antibody that recognizes both the MeCP2E1 isoform and the MeCP2E2 isoforms (Abcam Inc., Toronto, ON, CA) detected specific nuclear staining in the murine wild type (WT) hippocampus (FIGS. 10(A1), 10(A2), 10(A3)). As a negative control, the IHC experiments were performed in the null male Mecp2^{tm1.1Bird} y/− transgenic RTT mouse model wherein exons 3 and 4 of Mecp2 are deleted and Mecp2 transcripts and protein are non-detectable (Guy et al., 2001, A mouse Mecp2-null mutation causes neurological symptoms that mimic Rett syndrome. Nat. Genet. 27:322-326). The IHC experiments were repeated with the Mecp2^{tm1.1Bird} y/− null male mice at 4-6 weeks of age. MeCP2 signals were not detected (FIGS. 10(A4), 10(A5)), as expected.

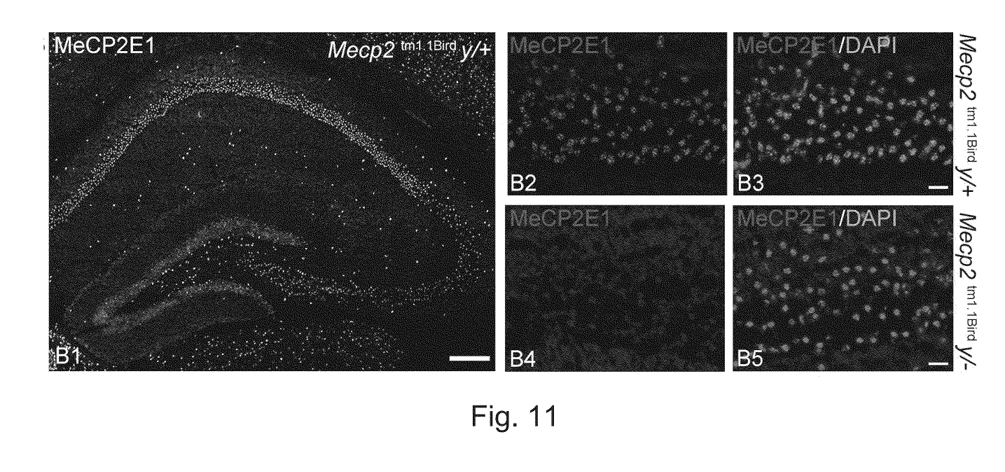

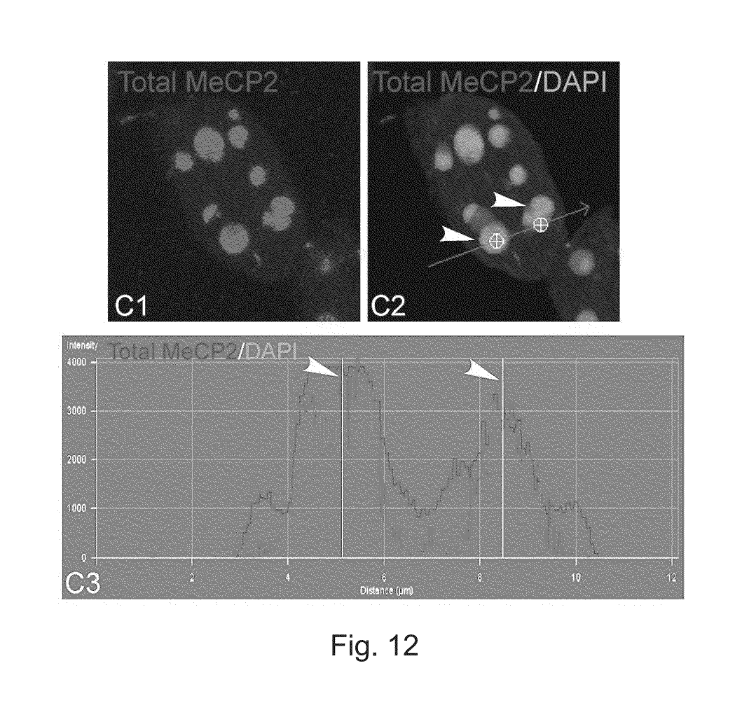

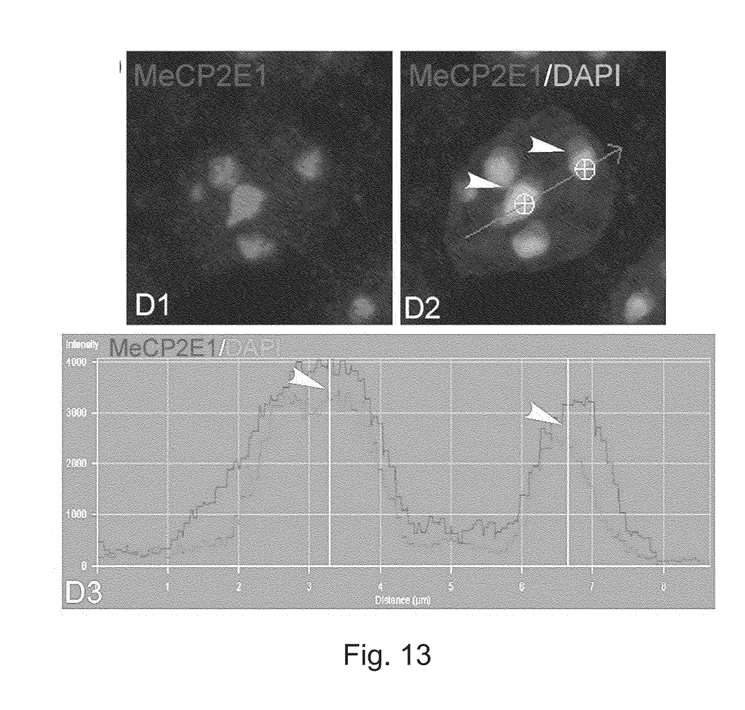

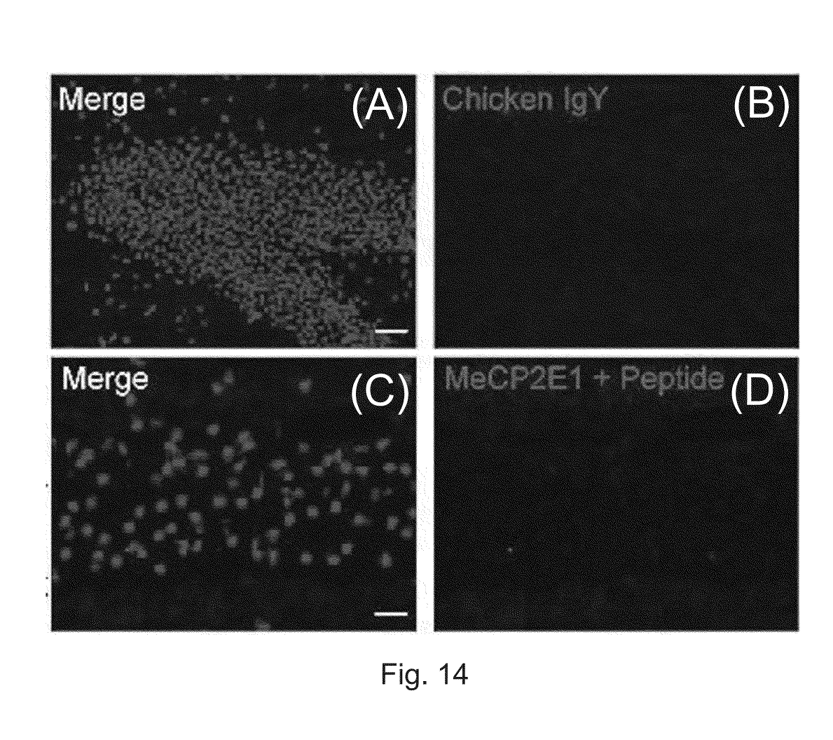

The next study assessed whether visible signals using the newly developed anti-MeCP2E1 antibody could be detected under similar conditions. Immunohistochemical experiments with the anti-MeCP2E1 antibody revealed nuclear MeCP2E1 signals in the adult WT hippocampus (FIGS. 11(B1), 11(B2), 11(B3)), with no detectable signals detected in the Mecp2^{tm1.1Bird} y/− null male mice (FIGS. 11(B4, 11(B5)). Confocal imaging and subsequent signal profile analysis revealed that MeCP2E1 expression had enriched localization at the DAPI-rich heterochromatic regions, similar to what is observed for total MeCP2 (FIGS. 12(C1)-12(C3): FIGS. 13(D1)-13(D3)). No signals were observed with negative controls when chicken IgY antibody was used instead of polyclonal chicken anti-MeCP2E1 antibody (FIGS. 14(A), 14(B)) or when anti-MeCP2E1 peptide competition was performed in hippocampal regions under identical experimental conditions (FIGS. 14(C), 14(D)).

Taken together, these results indicate that the newly developed anti-MeCP2E1 antibody specifically detects the endogenous murine MeCP2E1 and that MeCP2E1 shows similar nuclear localization compared to the total MeCP2 in the DAPI-rich heterochromatin regions of the nucleus.

### Example 9

### MeCP2E1 is Expressed at Different Levels within Different Regions of Murine Brain

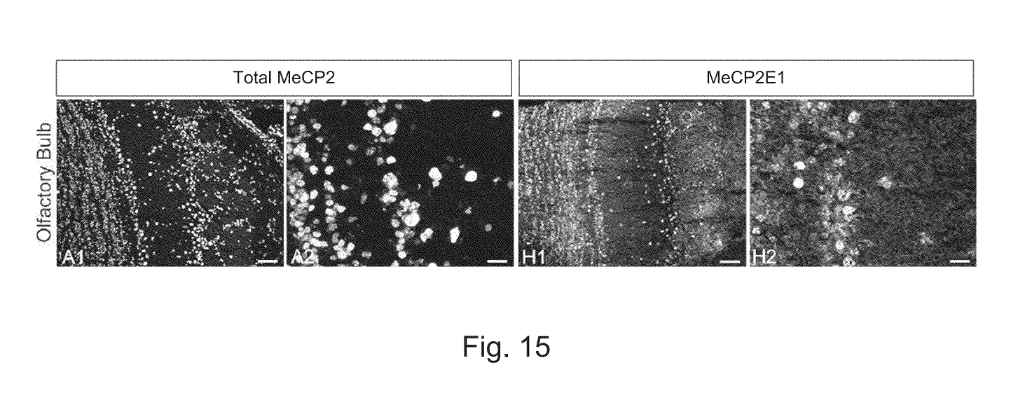

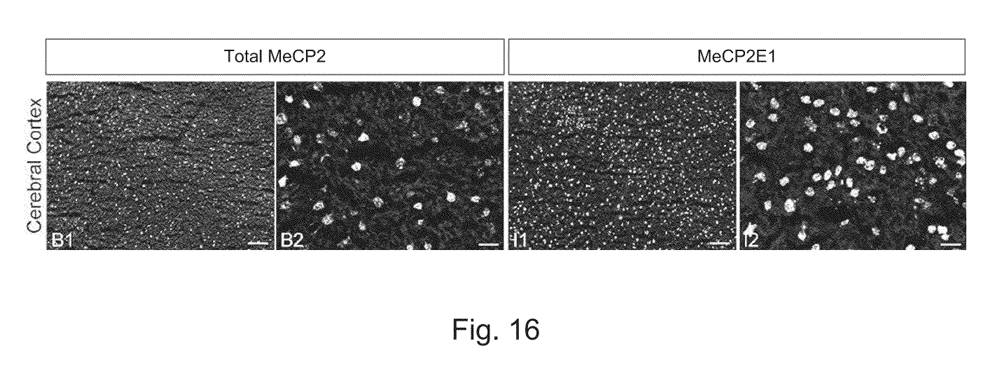

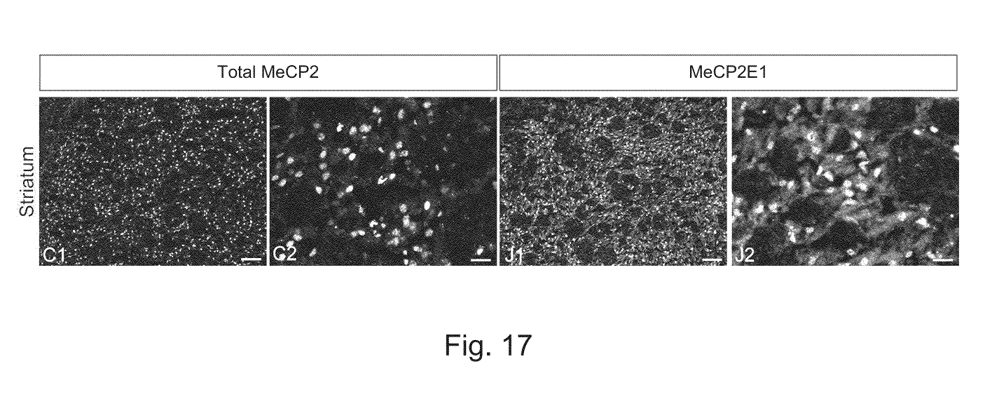

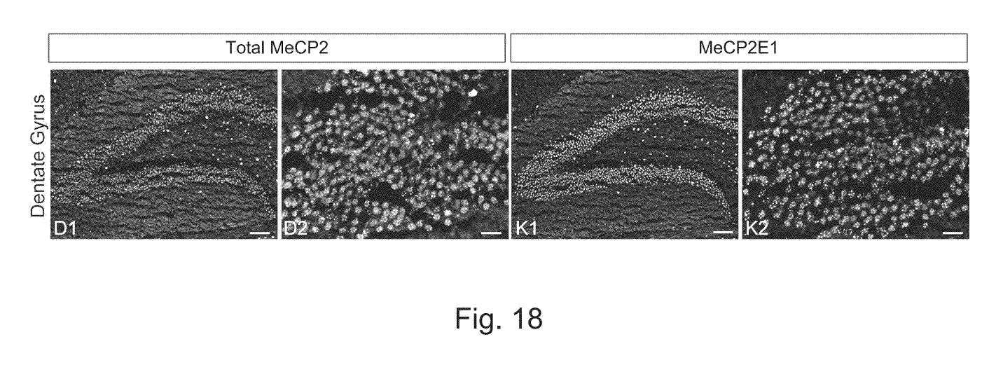

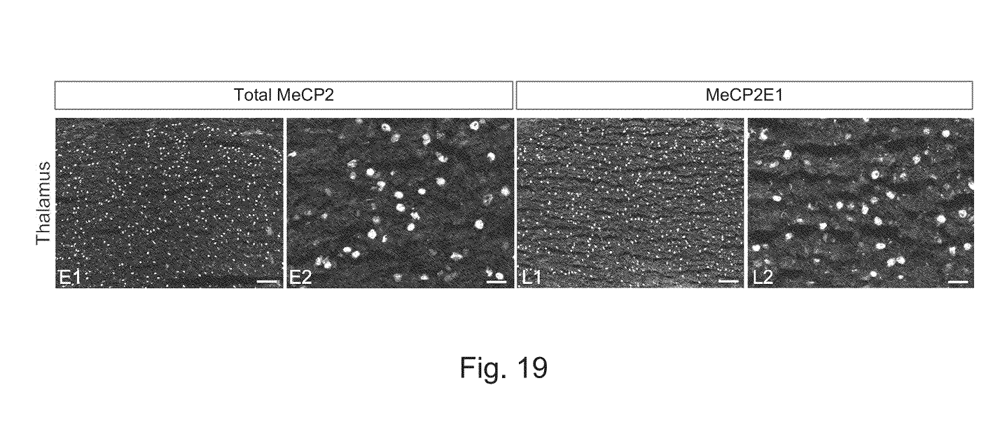

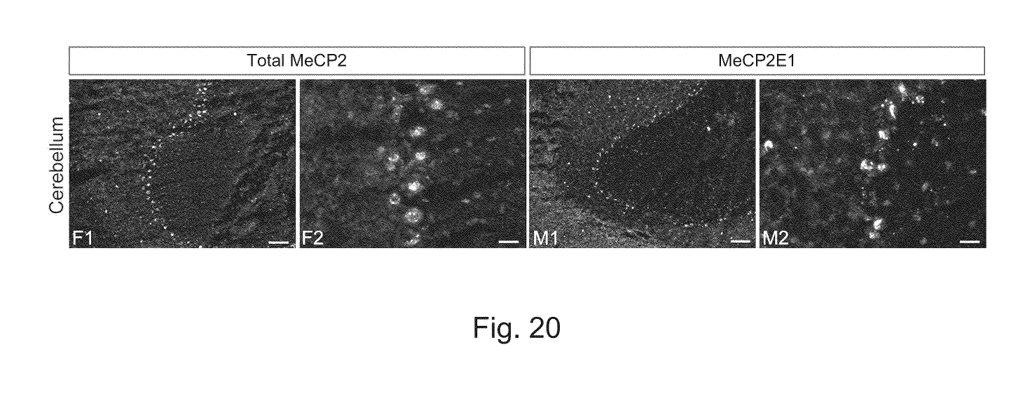

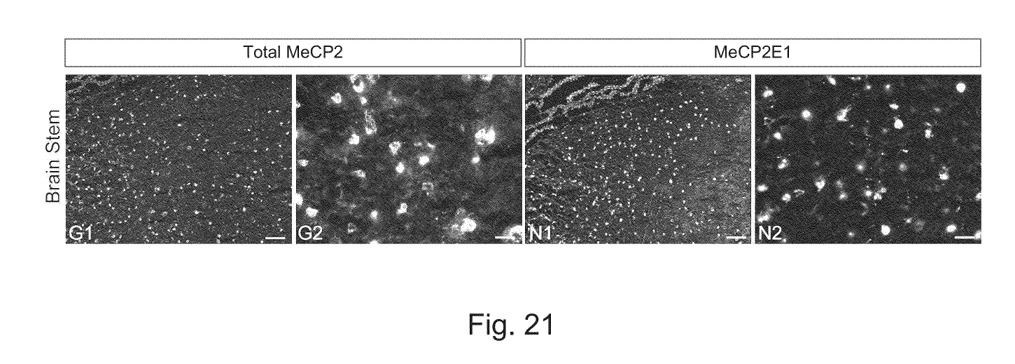

MeCP2 displays highest expression in brain, as compared to other tissues (Shahbazian et al., 2002; Thambirajah et al., 2012, MeCP2 binds to nucleosome free (linker DNA) regions and to H3K9/H3K27 methylated nucleosomes in the brain. Nucleic Acids Res. 40:2884-2897; Skene et al., 2010, Neuronal MeCP2 is expressed at near histone-octamer levels and globally alters the chromatin state. Mol. Cell. 37:457-468). Previous studies on Mecp2E1 and Mecp2E2 transcripts have reported variable transcript abundance in a brain region-specific manner (Dragich et al., 2007). Additionally, it is known that the transcript levels of Mecp2 and the corresponding protein expression are non-complementary (Shahbazian et al., 2002). This highlights the need to determine the expression of MeCP2E1 at the protein level. Therefore, the expression of MeCP2E1 was examined within various regions of the adult murine brain including olfactory bulb (FIGS. 15(H1) 15(H2)), cerebral cortex (FIGS. 16(I1) 16(I2)), striatum (FIGS. 17(J1) 17(J2)), dentate gyrus (FIGS. 18(K1) 18(K2)), thalamus (FIGS. 19(L1) 19(L2)), cerebellum (FIGS. 20(M1) 20(M2)), and brainstem (FIGS. 21(N1) 21(N2)). Total MeCP2 expression was detected with a commercial C-terminal antibody in the above-mentioned regions across the WT adult mice brain and as expected, MeCP2 expression was detectable in all tested brain regions and showed the characteristic nuclear MeCP2 signals (FIGS. 15(A1), 15(A2), 16(B1), 16(B2), 17(C1), 17(C2), 18(D1), 18(D2), 19(E1), 19(E2), 20(F1), 20(F2), 21(G1), 21(G2)). Immunolabelling with the anti-MeCP2E1 antibody demonstrated a broad distribution pattern of endogenous MeCP2E1 across all these studied regions of the murine brain (FIGS. 15(H1) 15(H2), 16(I1) 16(I2), 17(J1) 17(J2), 18(K1) 18(K2), 19(L1) 19(L2), 20(M1) 20(M2), 21(N1) 21(N2)).

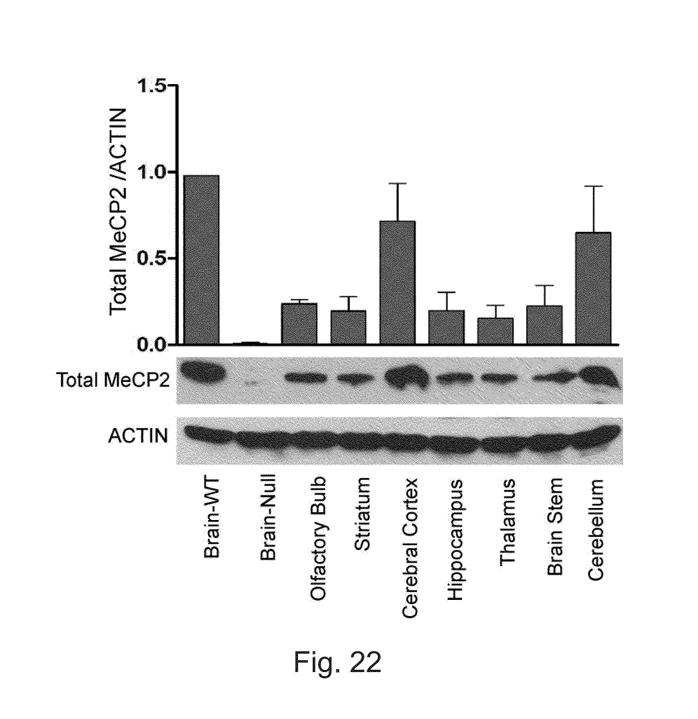

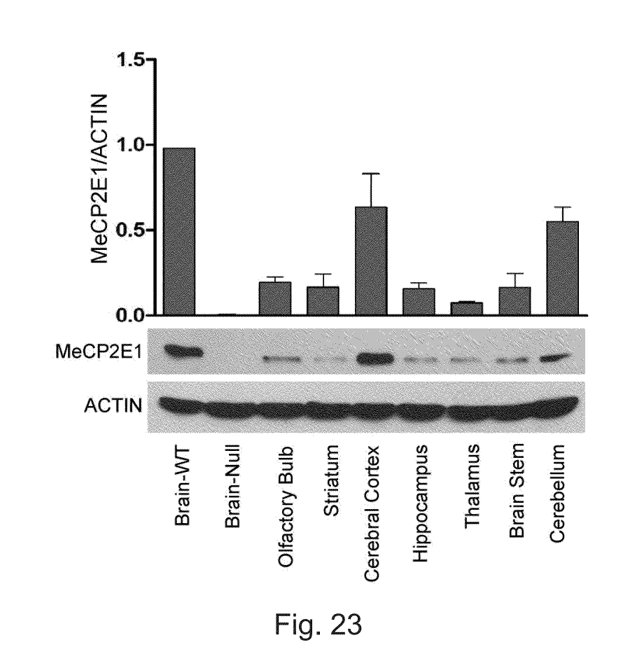

Quantitative analysis of total MeCP2 levels within these tested brain regions by WB demonstrated that the highest levels of total MeCP2 are detected in cerebral cortex and cerebellum as compared to other regions (FIG. 22). Analysis of MeCP2E1 levels in these regions, revealed a similar pattern (FIG. 23), suggesting that MeCP2E1 is likely the major MeCP2 isoform expressed in these regions.

Taken together, these data indicate that MeCP2E1 is predominantly and broadly expressed within the adult murine brain, with differential expression levels in various brain regions.

### Example 10

### MeCP2E1 has Higher Expression in Primary Neurons Compared with Primary Astrocytes

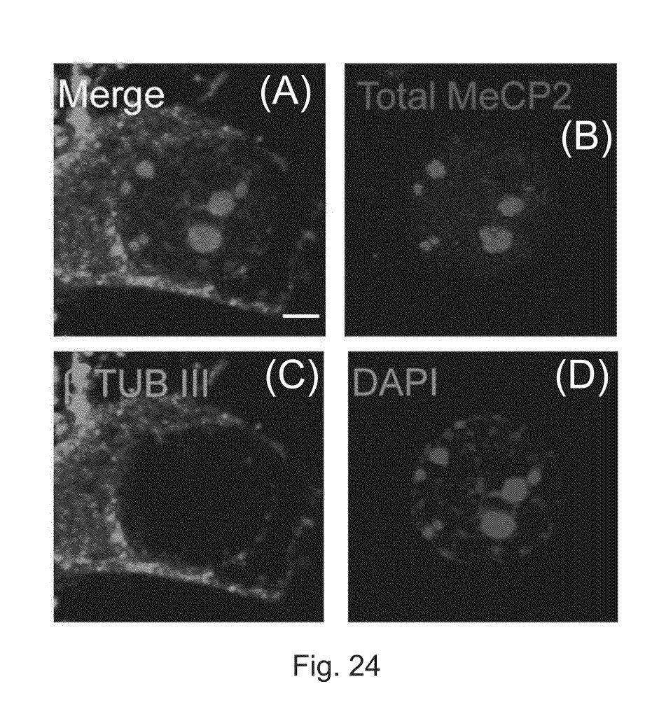

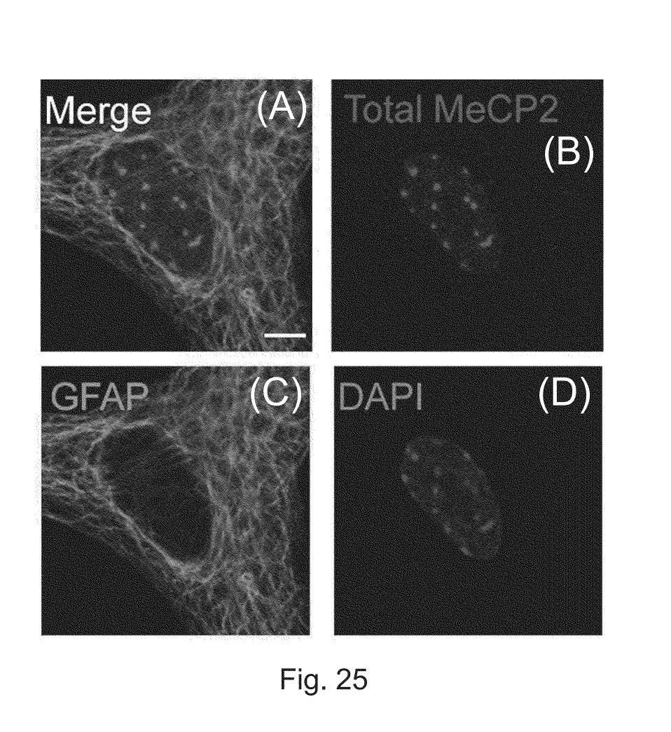

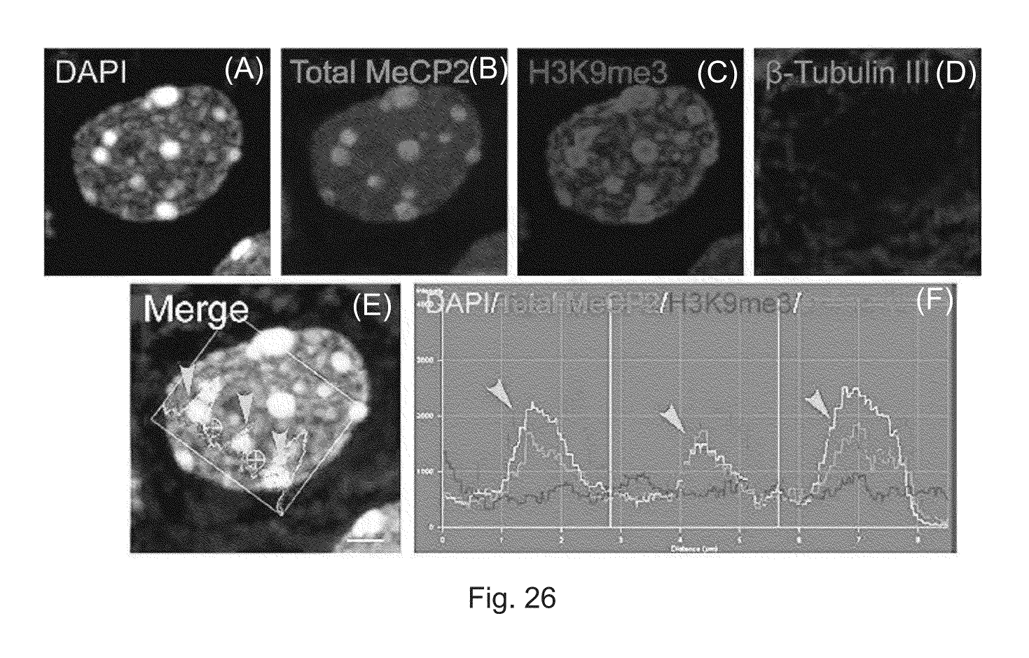

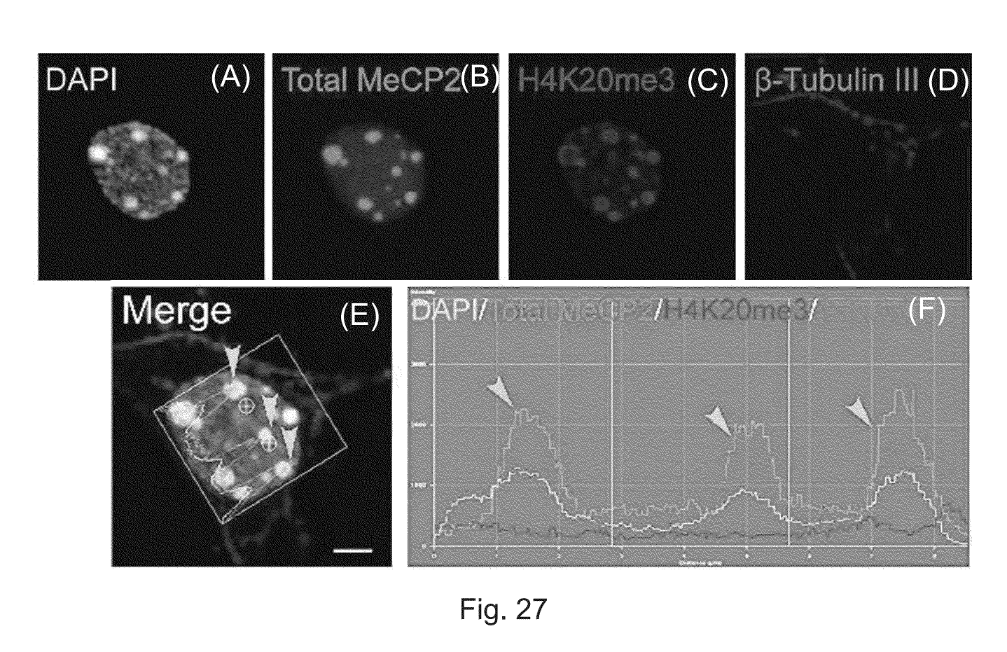

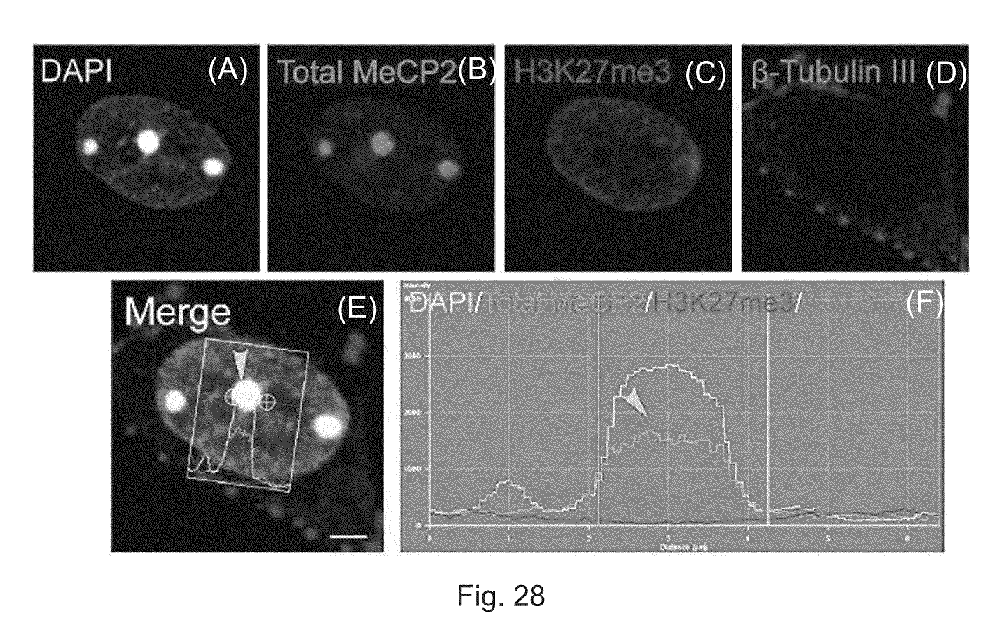

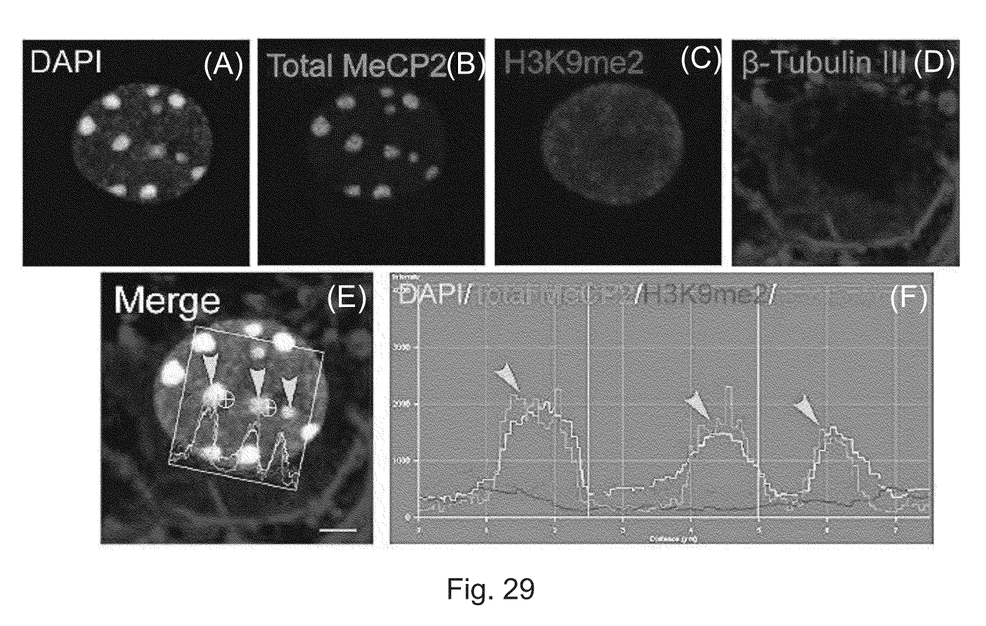

The expression of MeCP2 in astrocytes has been a relatively recent discovery, which has lead to a significant paradigm shift on the contribution of glial cells towards RTT pathophysiology. Re-expression of MeCP2 in astrocytes in RTT mice models mitigates many RTT phenotypes. However the expression of MeCP2 isoforms and their potential role in astrocyte function remain to be determined. Additionally, the expression of MeCP2 protein isoforms at the protein levels in neurons is still unknown. Therefore, the newly developed anti-MeCP2E1 antibody was used to examine the expression of MeCP2E1 in primary cortical neurons and astrocytes. As expected, the endogenous expression of total MeCP2 was detected in both primary cortical neurons (FIGS. 24(A)-24(D)) and astrocytes (FIGS. 25(A)-25(D)) using a C-terminal antibody. Previous studies have suggested that MeCP2 expression in primary neurons might vary from diffuse to punctuate staining within the nucleus based on culture conditions (Martinowich et al., 2003, DNA methylation-related chromatin remodeling in activity-dependent BDNF gene regulation. Science 302:890-893; Adachi et al., 2005, A segment of the Mecp2 promoter is sufficient to drive expression in neurons. Hum. Mol. Genet. 14:3709-3722). Therefore, the nuclear MeCP2 expression was examined in these primary neurons by confocal co-localization studies compared to constitutive and facultative heterochromatin marks (FIGS. 26(A)-26(F), 27(A)-27(F), 28(A)-28(F), 29(A)-29(F)). As shown in FIGS. 26(A)-26(F), 27(A)-27(F), 28(A)-287(F), 29(A)-29(F), MeCP2 is primarily co-localized with the two tested constitutive heterochromatin marks (H3K9me3, H4K20me3), but showed minimal overlapping pattern with the facultative heterochromatin marks (H3K27me3, H3K9me2).

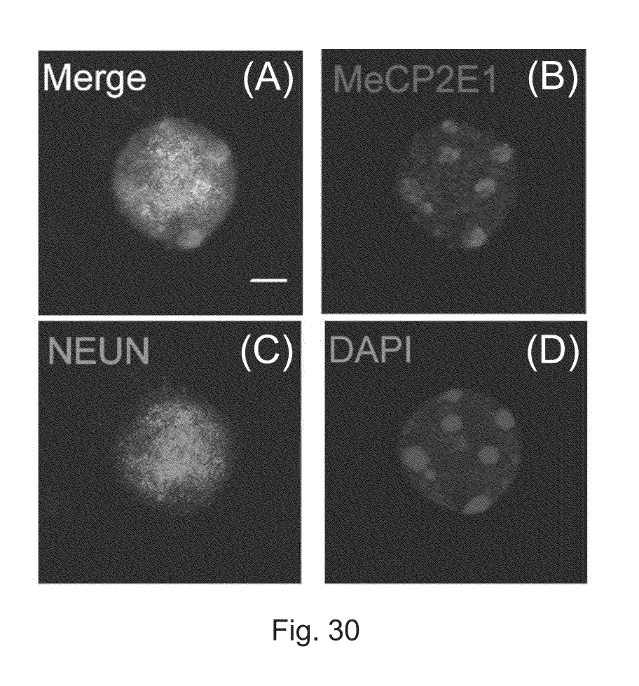

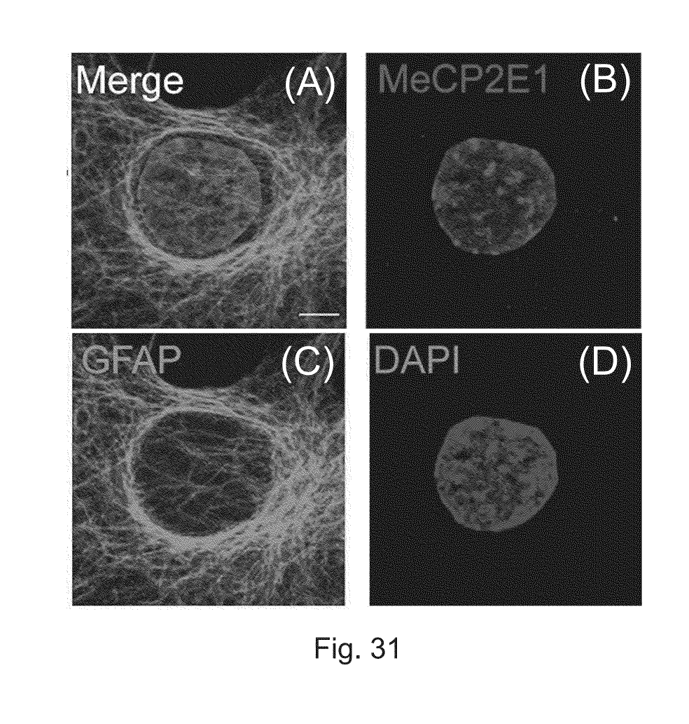

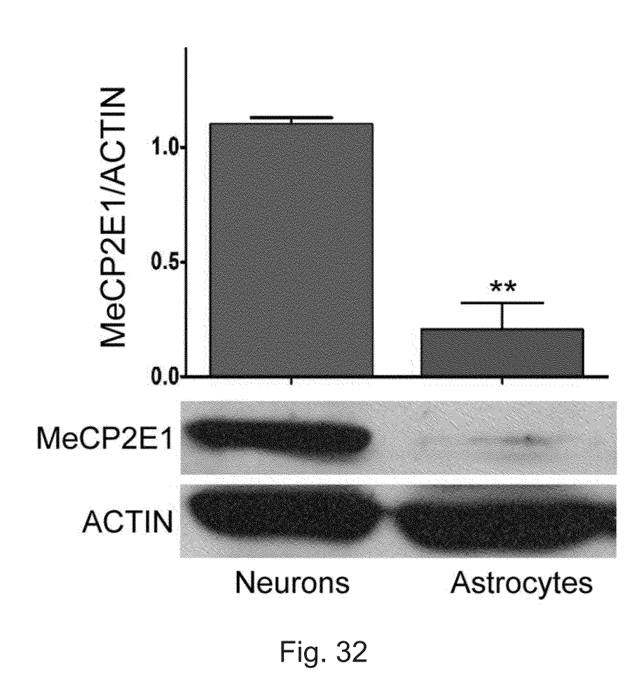

Immunofluorescence experiments with the newly generated anti-MeCP2E1 antibody detected endogenous MeCP2E1 expression in both primary neurons (FIGS. 30(A)-30(D)) and astrocytes (FIGS. 31(A)-31(D)) with nuclear heterochromatic expression pattern overlapping with DAPI signals. Although these data indicate that MeCP2E1 has similar nuclear localization compared to the total MeCP2 in both primary neurons and astrocytes, they do not reflect the protein levels in these two cell types. As a quantitative approach, the total amount of MeCP2E1 was examined in primary neurons and astrocytes by WB analysis and compared it to the ACTIN levels. It was found that that MeCP2E1 levels were indeed five times higher in primary neurons as compared to primary astrocytes (FIG. 32). This is not surprising, as primary astrocytes are reported to express approximately 25% of MeCP2 levels observed in primary neurons.

The anti-MeCP2E1 antibody produced with an antigen comprising a peptide with a sequence of eleven amino acids from the N-terminus of MeCP2E1 as disclosed herein, provides novel avenues for understanding brain region and/or cell type-specific expression of MeCP2E1 that will provide vital insights for the efficient design of future gene therapy approaches. Taken together, the results disclosed herein indicate that while MeCP2E1 is expressed in both primary cortical neurons and astrocytes, its level of expression is significantly higher in neurons. The data further indicate that in both primary neurons and astrocytes, MeCP2E1 signals highly overlap with DAPI-rich heterochromatin regions in the nucleus. Additionally, these data confirm that punctuated MeCP2 heterochromatic localization in neurons has significant overlap with constitutive heterochromatin marks, but has low overlap with the facultative heterochromatin marks. The higher abundance of MeCP2E1 in primary neurons compared to astrocytes suggests that MeCP2E1 might be significantly contributing towards the physiological symptoms associated with Rett syndrome.

1. An antibody that binds a MeCP2E1 isoform of Methyl CpG Binding Protein 2, wherein said antibody comprises a region comprising the amino acid sequence set forth in SEQ ID NO:4 or SEQ ID NO:5.

2. The antibody of claim 1, wherein said antibody does not bind a MeCP2E2 isoform of Methyl CpG Binding Protein 2.

3. The antibody of claim 1, wherein said antibody is a polyclonal antibody.

4. The antibody of claim 1, wherein said antibody is a monoclonal antibody.

5. The antibody of claim 1, wherein the antibody is conjugated to a detectable label.

6. The antibody of claim 5, wherein the label is a fluorescent label, an enzymatic label, a luminescent label, or a chromaphore label.

7. A composition comprising an antibody that binds a MeCP2E1 isoform of Methyl CpG Binding Protein 2, wherein said antibody comprises a region comprising the amino acid sequence set forth in SEQ ID NO:4.

8. The composition of claim 7, wherein said antibody does not bind a MeCP2E2 isoform of Methyl CpG Binding Protein 2.

9. The composition of claim 7, wherein said antibody is a polyclonal antibody.

10. The composition of claim 7, wherein the antibody is conjugated to a detectable label.

11. The composition of claim 7, wherein the label is a fluorescent label, an enzymatic label, a luminescent label, or a chromaphore label.

12. The composition of claim 7, wherein the antibody is packaged in a lyophilized form.

13. The composition of claim 7, wherein the antibody is packaged in an aqueous medium.

14. A peptide for use as an antigen to prepare the antibody of claim 1, wherein said peptide comprises a region comprising the amino acid sequence set forth in SEQ ID NO:4 or SEQ ID NO:5.

15. A kit detecting and/or monitoring an over-expression or an under-expression of a MeCP2E1 isoform of Methyl CpG Binding Protein 2, the kit comprising the composition of claim 6.

16. The kit of claim 15, wherein the kit additionally comprises a detectable label.

17. The kit of claim 16, wherein the detectable label is a fluorescent label, an enzymatic label, a luminescent label, or a chromaphore label.

18. The kit of claim 16, additionally comprising one or more compounds for detecting the label.

19. Use of any of claims 1, 7, and 15 for detecting and/or monitoring a disease or a disorder caused by an over-expression or an under-expression of a MeCP2E1 isoform of Methyl CpG Binding Protein 2.

20. A method for detecting and/or monitoring a disease or a disorder caused by an over-expression or an under-expression of a MeCP2E1 isoform of Methyl CpG Binding Protein 2, comprising the steps of:
obtaining a first sample from a mammalian subject;
contacting the first sample with an antibody that binds a MeCP2E1 isoform of Methyl CpG Binding Protein 2, wherein said antibody comprises a region comprising the amino acid sequence set forth in SEQ ID NO:4 or SEQ ID NO:5;
removing unbound antibody from the sample;
conducting an immunoassay on the first sample to determine a first value for expression of the MeCP2E1 isoform;
comparing the first value to a reference value for expression of the MeCP2E1 isoform in healthy mammalian subjects;
wherein a deviation of the first value from the reference value indicates the presence of a disease or a disorder caused by an over-expression or an under-expression of the MeCP2E1 isoform.

21. The method of claim 20, additionally comprising:
obtaining a second sample from a mammalian subject;
contacting the first sample with the antibody;
conducting an immunoassay on the second sample to determine a second value for expression of the MeCP2E1 isoform;
comparing the second value to one or both of the first value and the reference value;
wherein a deviation of the second value from one or both of the first value and the reference value indicates the change in the disease or the disorder caused by an over-expression or an under-expression of the MeCP2E1 isoform.

22. The method of claim 20, wherein the antibody is conjugated to a detectable label.

23. The method of claim 20, wherein the label is a fluorescent label, an enzymatic label, a luminescent label, or a chromaphore label.

24. The method of claim 20, wherein said antibody does not bind a MeCP2E2 isoform of Methyl CpG Binding Protein 2.
